(19) United States
(12) Patent Application Publication
(10) Pub. No.: US 2023/0095680 A1
(43) Pub. Date: Mar. 30, 2023

(54) COMPOSITIONS AND METHODS OF USING SUCH COMPOSITIONS AS AN ORAL RINSE

(71) Applicant: Jade Kim, Seattle, WA (US)

(72) Inventor: Jade Kim, Seattle, WA (US)

(21) Appl. No.: 17/478,635

(22) Filed: Sep. 17, 2021

(51) Int. Cl. A61K 8/49; A61K 8/19; A61Q 11/00

(52) U.S. Cl. CPC A61K 8/4966; A61K 8/19; A61Q 11/00

(57) ABSTRACT

The present disclosure provides compositions comprising sodium dichloroisocyanurate (NaDCC), or a functional analog thereof; and a sugar substitute, as well as methods for using such compositions to control bad breath or to improve oral health.

### TECHNICAL FIELD

The present disclosure is generally related to compositions comprising sodium dichloroisocyanurate and a sugar substitute for use in controlling bad breath or improving oral health, as well as methods of making and using the same. Additional methods for using compositions comprising sodium dichloroisocyanurate (NaDCC), either alone or together with other active components, to improve oral health are also disclosed.

### DESCRIPTION OF THE RELATED ART

Periodontal diseases include a broad spectrum of illnesses ranging from mild gingivitis to severe periodontitis. Virtually all types of periodontal diseases result from the interplay between pathogenic agents (e.g., bacteria, viruses, and yeast) and host immune responses. Following infection, inflammatory mediators, such as prostaglandins and interleukins, and enzymes, such as matrix metalloproteinases, are involved in the destruction of periodontal tissues. Eventually, a cascade of events leads to osteoclastogenesis and bone loss. Heritable and acquired environmental risk factors explain the susceptibility of certain individuals to periodontal diseases.

The major elements of current periodontal therapy are mechanical pocket debridement, periodontal pocket irrigation with potent antiseptics, treatment of advanced disease with systemic antibiotics and attention to proper self-care (Slots, J., Low-cost periodontal therapy. Periodontol 2000 2012:60:110-137).

The worldwide increase of antibiotic-resistant bacteria and the high cost of new, effective antibiotics have increased a need for an effective and safe antiseptic to combat surface infections, such as those of the periodontium.

Previously, antiseptic oral rinses have included, for example, dilute solutions of sodium hypochlorite (NaOCl), which is found in liquid bleach. However, NaOCl provides serious safety concerns if stored or used improperly. NaOCl is generally sold as a solution and must be diluted before being used as an oral rinse. If not properly diluted, it can cause skin irritation and damage to the surface of the teeth. Further, NaOCl decomposes to form chlorite and/or chlorate ions, which are undesirable as they have been found to cause oxidative stress or damage to red blood cells. Thus, the shelf life of products that include NaOCl (such as liquid bleach) is limited, and the concentration of NaOCl in a given solution may change considerably over time.

Therefore, there is a need for improved compositions and methods for improving oral health. The presently disclosed embodiments address this need and provide other related advantages.

### BRIEF SUMMARY

As noted herein, the present disclosure provides for compositions comprising sodium dichloroisocyanurate (NaDCC), or a functional analog thereof; and a sugar substitute that are useful in controlling bad breath or improving oral health, as well as methods of making and using the same. Additional methods for using compositions comprising sodium dichloroisocyanurate (NaDCC), either alone or together with other active components, to control bad breath or improve oral health are also disclosed.

In aspects, the present disclosure includes a composition, comprising NaDCC, or a functional analog thereof; a sugar substitute selected from Stevia, aspartame, xylitol, or sorbitol, or a functional analog thereof, or a combination thereof; an alkali metal carbonate selected from calcium carbonate, calcium bicarbonate, sodium carbonate, sodium bicarbonate, or a combination thereof; and an acid selected from adipic acid, citric acid, tartaric acid, or a combination thereof.

In further aspects, the present disclosure provides a method of manufacturing a composition of the disclosure, the method comprising combining the NaDCC, or the functional analog thereof, the sugar substitute, the alkali metal carbonate, and the acid into a mixture; and homogenizing the mixture.

The present disclosure further includes a method of controlling bad breath in a subject, the method comprising contacting at least a portion of an oral cavity of the subject with an effective amount of a composition of the disclosure.

The present disclosure further includes a method of improving oral health in a subject, the method comprising contacting at least a portion of an oral cavity of the subject with an effective amount of a composition of the disclosure.

In aspects, the present disclosure includes methods for using compositions comprising sodium dichloroisocyanurate (NaDCC), either alone or together with other active components, such as an alkali carbonate, to improve oral health.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1A and FIG. 1B are photographs of symptoms of periodontal disease in Patient 1. Patient 1 used an oral rinse of the disclosure as directed for one month. FIG. 1A depicts the symptoms of periodontal disease prior to treatment. FIG. 1B depicts the condition after treatment.

FIG. 2A and FIG. 2B are photographs of symptoms of periodontal disease in Patient 2. Patient 2 used an oral rinse of the disclosure as directed for two weeks. FIG. 2A shows the symptoms of periodontal disease prior to treatment. FIG. 2B shows the condition after treatment.

FIG. 3A and FIG. 3B are photographs of symptoms of periodontal disease in Patient 3. Patient 3 used an oral rinse of the disclosure as directed for two weeks. FIG. 3A shows the symptoms of periodontal disease prior to treatment. FIG. 3B shows the condition after treatment.

FIG. 4A and FIG. 4B are photographs of symptoms of periodontal disease in Patient 4. Patient 4 used an oral rinse of the disclosure as directed for two months. FIG. 4A shows the symptoms of periodontal disease prior to treatment. FIG. 4B shows the condition after treatment.

FIG. 5A-1, FIG. 5A-2, FIG. 5B-1, and FIG. 5B-2 are periodontal charts from the same patient before and after treatment with a composition of the disclosure. The patient used the composition on a daily basis for 11 months. FIG. 5A-1 and FIG. 5A-2 are a periodontal chart completed based on an examination prior to treatment with the composition. FIG. 5B-1 and FIG. 5B-2 are a periodontal chart for the same patient completed based on an examination after treatment.

FIG. 6A and FIG. 6B provide the results of pH testing on compositions with varying amounts of xylitol, as well as on a control composition.

FIG. 7 is photographs of unprocessed and milled xylitol.

FIG. 8 illustrates the average time difference between dissolution and disintegration for compositions with varying amounts of xylitol.

FIG. 9 illustrates the average dissolution and disintegration times for compositions with varying amounts of xylitol in either unprocessed or milled form.

FIG. 10 provides the results dissolution and disintegration testing for compositions with varying amounts of xylitol formulated in several tablet sizes.

### DETAILED DESCRIPTION

The present disclosure provides compositions for improving oral health, more specifically, compositions comprising sodium dichloroisocyanurate (NaDCC), or a functional analog thereof; and xylitol, or a functional analog thereof. Such compositions are antiseptic, and may be used to reduce periodontal disease. The present disclosure further provides for methods of manufacturing and using the compositions.

In the following description, certain specific details are set forth in order to provide a thorough understanding of various embodiments of the disclosure. However, one skilled in the art will understand that the disclosure may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “consisting essentially of” mean±20% of the indicated range, value, or structure, unless otherwise indicated. The use of the alternative (e.g., “or”) should be understood to mean either one, both, or any combination thereof of the alternatives.

“Optional” or “optionally” means that the subsequently described element, component, event, or circumstance may or may not occur, and that the description includes instances in which the element, component, event, or circumstance occurs and instances in which they do not.

“Functional analog” refers to a compound that is structurally similar or substantially structurally similar to a parent or reference compound of the disclosure, but differs slightly in composition (e.g., one atom or functional group is different, added, or removed), such that it is capable of performing at least one function of the referenced compound with at least 90% efficacy. In other words, a functional analog of a compound of the disclosure will have no more than a 10% reduction in performance of at least one function as compared to the compound of the disclosure when assayed using a technique known to one of skill in the art.

An “analog” may or may not have different chemical or physical properties than the original compound and may or may not have improved biological or chemical activity. For example, an analog may be more hydrophilic or it may have altered activity as compared to a parent compound. An analog may mimic the chemical or biological activity of the parent compound (i.e., it may have similar or identical activity), or, in some cases, may have increased or decreased activity. In other cases, the changes in an analog may impart certain desirable properties (e.g., improved stability, improved bioavailability, improved hydrophilicity, minimized off-target effects, minimized toxicity). An analog may be a naturally or non-naturally occurring (e.g., chemically-modified) variant of the original compound. In some embodiments, an analog of a compound of the disclosure is a pharmaceutically acceptable salt, polymorph, solvate, or tautomer of the compound.

“Pharmaceutically acceptable salts” include both acid and base addition salts. Even if not specifically described in each instance, unless otherwise indicated (e.g., by the context), use of a therapeutic agent described herein optionally comprises use of a pharmaceutically acceptable salt of the therapeutic agent instead of, or in addition to, the parent compound.

“Pharmaceutically acceptable acid addition salt” refers to those salts which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid (e.g., L-(+)-tartaric acid), thiocyanic acid, p-toluenesulfonic acid, trifluoroacetic acid, undecylenic acid, and the like.

“Pharmaceutically acceptable base addition salt” refers to those salts which are prepared from addition of an inorganic base or an organic base to a free acid. Salts derived from inorganic bases include the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“Polymorph” refers to a crystalline form of a compound with a distinct spatial lattice arrangement as compared to other crystalline forms of the same compound. A polymorph may also be defined as different unsolvated crystal forms of a compound. Specific polymorphs can have different chemical and physical properties. Unless otherwise specifically noted, the term polymorph is intended to encompass pseudopolymorphs as well.

“Pseudopolymorph” refers to a polymorph that incorporates a solvent or water into the crystal lattice of the compound. Specific examples of pseudopolymorphs encompassed by the invention include solvates (which are understood to be crystalline forms incorporating solvent into the crystal lattice of the compound) and hydrates (which are understood to mean crystalline forms incorporating water in the crystal lattice of the compound). A pseudopolymorph may also be a desolvated solvate (i.e., a form that can be made by removing the solvent from a solvate) or a dehydrated hydrate (i.e., a form that can be made by removing the water from a hydrate).

“Solvate” refers to a compound formed by solvation, for example as a combination of solvent molecules with molecules or ions of a solute. Well known solvent molecules include water, alcohols and other polar organic solvents. Alcohols include methanol, ethanol, n-propanol, isopropanol, n-butanol, isobutanol, and t-butanol. Alcohols also include polymerized alcohols such as polyalkylene glycols (e.g., polyethylene glycol, polypropylene glycol). The best-known and preferred solvent is typically water, and solvate compounds formed by solvation with water are termed hydrates.

“Disintegration time” refers to the length of the effervescent reaction, i.e., the period of time starting from the beginning of an effervescent reaction until the effervescent reaction ceases.

“Dissolution time” refers to the period of time needed for a tablet to completely pass into solution.

“Tautomer” refers to constitutional isomers of chemical compounds that readily interconvert.

“Subject” includes humans, domestic animals, such as laboratory animals, household pets, and livestock (e.g., cats, dogs, swine, cattle, sheep, goats, horses, rabbits, etc.), and non-domestic animals (e.g., bears, large cats, raccoons, deer, beavers, squirrels, etc.).

“Bad breath” refers to a persistent, unpleasant odor in the exhaled breath of a subject. Bad breath may be caused by a variety of factors, including poor dental hygiene, bacteria, and periodontal disease. As used herein, “controlling” bad breath refers to the temporary reduction and/or elimination of bad breath (e.g., by masking the smell). In some embodiments, controlling bad breath further includes providing a pleasant taste or smell.

“Antiseptic” refers to a compound or composition that is effective against a broad-spectrum of microbial agents, and that is applied topically on living tissue to prevent clinical infections by bacteria and viruses.

“Periodontal disease” refers to a pathological inflammatory condition of the gums and bone support surrounding teeth. Periodontal disease includes gingivitis, which is inflammation of the gums at the necks of the teeth, and periodontitis, which is inflammation affecting the bone and tissues of the teeth. Periodontal disease can be caused by pathogenic agents, trauma, and host immune response, or any combination thereof. In particular embodiments, periodontal disease is caused at least in part by bacterial biofilm.

“Treating” or “treatment” as used herein refers to the treatment of periodontal disease in a subject, such as a human, having periodontal disease, and includes: (i) preventing periodontal disease from occurring in a mammal, in particular, when such mammal is predisposed to periodontal disease but has not yet been diagnosed as having it; (ii) inhibiting periodontal disease, i.e., arresting its development; (iii) relieving the periodontal disease, i.e., causing regression of periodontal disease; or (iv) relieving the symptoms resulting from periodontal disease, (e.g., reducing dental plaque, etc.) without addressing the underlying periodontal disease.

“Effective amount” refers to the amount of a compound or composition of this disclosure which, when administered to a subject, such as a human, is sufficient to effect treatment. The amount of a compound or composition of this disclosure that constitutes an “effective amount” will vary depending on the compound or composition, the condition being treated and its severity, the manner of administration, the duration of treatment, or the age of the mammal to be treated, but can be determined routinely by one of ordinary skill in the art based on his own knowledge and this disclosure. In embodiments, an “effective amount” effects treatment (e.g., treats, promotes, improves, increases, reduces, suppresses, and the like) as measured by a statistically significant change in one or more indications, symptoms, signs, diagnostic tests, vital signs, and the like. In other embodiments, an “effective amount” manages or prevents a condition as measured by a lack of a statistically significant change in one or more indications, symptoms, signs, diagnostic tests, vital signs, and the like.

As used herein, “statistically significant” refers to a p value of 0.050 or less when calculated using the Students t-test and indicates that it is unlikely that a particular event or result being measured has arisen by chance.

Unless the context requires otherwise, throughout the present specification and claims, the word “comprise” and variations thereof, such as, “comprises” and “comprising” are to be construed in an open, inclusive sense (i.e., as “including, but not limited to”).

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an”, and “the” include plural references unless the context clearly dictates otherwise.

### Compositions

As noted herein, the present disclosure provides for compositions comprising sodium dichloroisocyanurate (NaDCC) or a functional analog thereof; and a sugar substitute (e.g., xylitol, Stevia, sorbitol, or aspartame, or a functional analog thereof).

“Sodium dichloroisocyanurate” and “NaDCC” refers to a compound having the following structure:

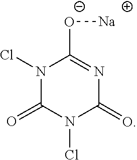

Generally, NaDCC is an antiseptic and a biocidal agent. In some embodiments, a composition of the present disclosure comprises NaDCC. In some embodiments, a composition of the present disclosure comprises a functional analog of NaDCC. In various embodiments, a functional analog of NaDCC will retain at least 90% of the antiseptic activity, biocidal activity, or both, as compared to NaDCC. In specific embodiments, the functional analog is the NaDCC dehydrate. In various embodiments, a composition of the present disclosure comprises NaDCC or a polymorph, solvate, or tautomer thereof.

In various embodiments, a composition of the disclosure comprises NaDCC, or a functional analog (e.g., a polymorph, solvate, or tautomer) thereof, in an amount ranging from 10 weight percent (wt. %) to 20 wt. %. In some embodiments, a composition of the disclosure comprises NaDCC, or a functional analog thereof, in an amount ranging from 11 wt. % to 20 wt. %. In specific embodiments, a composition of the disclosure comprises NaDCC, or a functional analog thereof, in an amount of about 13 wt. %. In specific embodiments, a composition of the disclosure comprises NaDCC, or a functional analog thereof, in an amount of about 14 wt. %. In specific embodiments, a composition of the disclosure comprises NaDCC, or a functional analog thereof, in an amount of about 15 wt. %. In specific embodiments, a composition of the disclosure comprises NaDCC, or a functional analog thereof, in an amount of about 16 wt. %. In specific embodiments, a composition of the disclosure comprises NaDCC, or a functional analog thereof, in an amount of about 17 wt. %.

In various embodiments, a composition of the disclosure comprises NaDCC, or a functional analog thereof, in an amount ranging from 15 wt. % to 60 wt. %. In some embodiments, a composition of the disclosure comprises NaDCC, or a functional analog hereof, in an amount ranging from 20 wt. % to 50 wt. %. In specific embodiments, a composition of the disclosure comprises NaDCC, or a functional analog thereof, in an amount of about 40 wt. %.

In various embodiments, a composition of the present disclosure comprises NaDCC, or a functional analog thereof, in an amount ranging from 5 milligrams (mg) to 11 mg. In some embodiments, a composition of the present disclosure comprises NaDCC, or a functional analog thereof, in an amount ranging from 5.5 mg to 11 mg. In some embodiments, a composition of the present disclosure comprises NaDCC, or a functional analog thereof, in an amount ranging from 7 mg to 10 mg. In specific embodiments, a composition of the present disclosure comprises about 7 mg of NaDCC, or a functional analog thereof. In specific embodiments, a composition of the present disclosure comprises about 7.5 mg of NaDCC, or a functional analog thereof. In specific embodiments, a composition of the present disclosure comprises about 8 mg of NaDCC, or a functional analog thereof. In specific embodiments, a composition of the present disclosure comprises about 8.5 mg of NaDCC, or a functional analog thereof. In specific embodiments, a composition of the present disclosure comprises about 9 mg of NaDCC, or a functional analog thereof. In specific embodiments, a composition of the present disclosure comprises about 9.5 mg of NaDCC, or a functional analog thereof. In specific embodiments, a composition of the present disclosure comprises about 10 mg of NaDCC, or a functional analog thereof.

In various embodiments, a composition of the present disclosure comprises NaDCC, or a functional analog thereof, in an amount ranging from 5 mg to 200 mg. In some embodiments, a composition of the present disclosure comprises NaDCC, or a functional analog thereof, in an amount ranging from 10 mg to 150 mg. In other embodiments, a composition of the present disclosure comprises NaDCC, or a functional analog thereof, in an amount ranging from 20 mg to 100 mg. In further embodiments, a composition of the present disclosure comprises NaDCC, or a functional analog thereof, in an amount ranging from 30 mg to 75 mg. In specific embodiments, a composition of the present disclosure comprises about 40 mg of NaDCC, or a functional analog thereof.

The term “sugar substitute” refers to a food additive that provides a sweet taste like that of sugar while containing significantly less food energy than sugar-based sweeteners, making it a zero-calorie or low-calorie sweetener. In particular, a sugar substitute is a sweetener with no more than four calories per gram. Sugar substitutes include allulose, acesulfame potassium, aspartame, cyclamate, mogrosides, saccharin, Stevia, sucralose, and sugar alcohols (e.g., sorbitol, xylitol, mannitol, erythritol, and lactitol). Advantageously, sugar substitutes have significantly reduced tooth erosion as compared to sugar.

In embodiments, the sugar substitute comprises Stevia, aspartame, or a sugar alcohol, or a functional analog thereof (e.g., a pharmaceutically acceptable salt, a polymorph, solvate, or tautomer), or a combination thereof. In embodiments, the sugar substitute comprises Stevia, aspartame, or a sugar alcohol, or a functional analog thereof (e.g., a pharmaceutically acceptable salt, a polymorph, solvate, or tautomer). In embodiments, the sugar substitute is Stevia, aspartame, or a sugar alcohol, or a functional analog thereof, or a combination thereof. In embodiments, the sugar substitute is Stevia, aspartame, or a sugar alcohol, or a functional analog thereof. In particular embodiments, the sugar alcohol is xylitol or sorbitol. Accordingly, in some embodiments, the sugar substitute comprises Stevia, aspartame, xylitol, or sorbitol, or a functional analog thereof. In some embodiments, the sugar substitute is Stevia, aspartame, xylitol, or sorbitol, or a functional analog thereof.

“Stevia” refers to a natural sweetener derived from the leaves of the plant species Stevia rebaudiana. The active compounds of Stevia are steviol glycosides. Stevitol has the following structure:

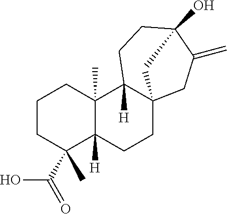

In some embodiments, a composition of the present disclosure comprises Stevia. In some embodiments, a composition of the present disclosure comprises a functional analog of Stevia. In various embodiments, a functional analog of Stevia will retain at least 90% of the sweetness as compared to Stevia. In various embodiments, a composition of the present disclosure comprises Stevia, or a pharmaceutically acceptable salt, polymorph, solvate, or tautomer thereof.

“Aspartame” refers to an artificial non-saccharide sweetener that is a methyl ester of an aspartic acid/phenylalanine dipeptide. Aspartame has the following structure:

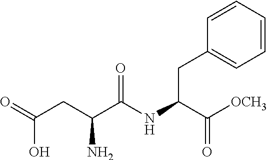 [SMILES: COC(=O)[C@H](Cc1ccccc1)NC(=O)[C@@H](N)CC(=O)O]

In some embodiments, a composition of the present disclosure comprises aspartame. In some embodiments, a composition of the present disclosure comprises a functional analog of aspartame. In various embodiments, a functional analog of aspartame will retain at least 90% of the sweetness as compared to aspartame. In various embodiments, a composition of the present disclosure comprises aspartame, or a pharmaceutically acceptable salt, polymorph, solvate, or tautomer thereof.

“Sugar alcohol” refers to an organic compound derived from a sugar and containing one hydroxyl group attached to each carbon atom. Sugar alcohols have the general formula HOCH₂(CHOH)ₙCH₂OH, and include sorbitol, xylitol, mannitol, erythritol, and lactitol. In some embodiments, a composition of the present disclosure comprises a sugar alcohol. In some embodiments, a composition of the present disclosure comprises a functional analog of a sugar alcohol. In various embodiments, a functional analog of a sugar alcohol will retain at least 90% of the sweetness as compared to the sugar alcohol. In various embodiments, a composition of the present disclosure comprises a sugar alcohol, or a pharmaceutically acceptable salt, polymorph, solvate, or tautomer thereof.

For example, “sorbitol” refers to a six-carbon sugar alcohol having the following structure:

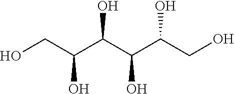 [SMILES: OC[C@@H](O)[C@@H](O)[C@H](O)[C@@H](O)CO]

In some embodiments, a composition of the present disclosure comprises sorbitol. In some embodiments, a composition of the present disclosure comprises a functional analog of sorbitol. In various embodiments, a functional analog of sorbitol will retain at least 90% of the sweetness as compared to sorbitol. In various embodiments, a composition of the present disclosure comprises sorbitol, or a pharmaceutically acceptable salt, polymorph, solvate, or tautomer thereof.

As an additional example, “xylitol” refers to a five-carbon sugar polyalcohol with a formula CH₂OH(CHOH)₃CH₂OH and having the following structure:

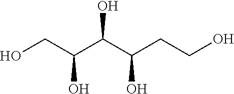 [SMILES: OCC[C@@H](O)[C@H](O)[C@@H](O)CO]

Xylitol, among other things, reduces dental caries, plaque, and bacterial biofilm. In some embodiments, a composition of the present disclosure comprises xylitol. In some embodiments, a composition of the present disclosure comprises a functional analog of xylitol. In various embodiments, a functional analog of xylitol will retain at least 90% of the activity that reduces dental caries, plaque, and bacterial biofilm, as compared to xylitol. In various embodiments, a functional analog of xylitol will retain at least 90% of the sweetness as compared to xylitol. In various embodiments, a composition of the present disclosure comprises xylitol, or a pharmaceutically acceptable salt, polymorph, solvate, or tautomer thereof. In some embodiments, the xylitol is present in a milled form.

In embodiments, a composition of the present disclosure comprises a sugar substitute in an amount ranging from 0.5 wt. % to 60 wt. %. In some embodiments, a composition of the disclosure comprises a sugar substitute in an amount ranging from 5 wt. % to 50 wt. %. In other embodiments, a composition of the disclosure comprises a sugar substitute in an amount ranging from 10 wt. % to 40 wt. %. In further embodiments, a composition of the disclosure comprises a sugar substitute in an amount ranging from 20 wt. % to 30 wt. %. In specific embodiments, a composition comprises a sugar substitute in an amount of about 26 wt. %. In further embodiments, a composition of the disclosure comprises a sugar substitute in an amount ranging from 2 wt. % to 10 wt. %. In specific embodiments, a composition comprises a sugar substitute in an amount of about 2.5 wt. %. In specific embodiments, a composition comprises a sugar substitute in an amount of about 3 wt. %. In specific embodiments, a composition comprises a sugar substitute in an amount of about 3.5 wt. %. In specific embodiments, a composition comprises a sugar substitute in an amount of about 4 wt. %. In specific embodiments, a composition comprises a sugar substitute in an amount of about 4.5 wt. %. In specific embodiments, a composition comprises a sugar substitute in an amount of about 5 wt. %. In specific embodiments, a composition comprises a sugar substitute in an amount of about 5.5 wt. %. In specific embodiments, a composition comprises a sugar substitute in an amount of about 6 wt. %. In specific embodiments, a composition comprises a sugar substitute in an amount of about 6.5 wt. %.

In embodiments, a composition of the present disclosure comprises Stevia, aspartame, or a sugar alcohol, or a functional analog thereof, in an amount ranging from 0.5 wt. % to 60 wt. %. In some embodiments, a composition of the disclosure comprises Stevia, aspartame, or a sugar alcohol, or a functional analog thereof, in an amount ranging from 5 wt. % to 50 wt. %. In other embodiments, a composition of the disclosure comprises Stevia, aspartame, or a sugar alcohol, or a functional analog thereof, in an amount ranging from 10 wt. % to 40 wt. %. In further embodiments, a composition of the disclosure comprises Stevia, aspartame, or a sugar alcohol, or a functional analog thereof, in an amount ranging from 20 wt. % to 30 wt. %. In specific embodiments, a composition comprises Stevia, aspartame, or a sugar alcohol, or a functional analog thereof, in an amount of about 26 wt. %. In further embodiments, a composition of the disclosure comprises Stevia, aspartame, or a sugar alcohol, or a functional analog thereof, in an amount ranging from 2 wt. % to 10 wt. %. In specific embodiments, a composition comprises Stevia, aspartame, or a sugar alcohol, or a functional analog thereof, in an amount of about 2.5 wt. %. In specific embodiments, a composition comprises Stevia, aspartame, or a sugar alcohol, or a functional analog thereof, in an amount of about 3 wt. %. In specific embodiments, a composition comprises Stevia, aspartame, or a sugar alcohol, or a functional analog thereof, in an amount of about 3.5 wt. %. In specific embodiments, a composition comprises Stevia, aspartame, or a sugar alcohol, or a functional analog thereof, in an amount of about 4 wt. %. In specific embodiments, a composition comprises Stevia, aspartame, or a sugar alcohol, or a functional analog thereof, in an amount of about 4.5 wt. %. In specific embodiments, a composition comprises Stevia, aspartame, or a sugar alcohol, or a functional analog thereof, in an amount of about 5 wt. %. In specific embodiments, a composition comprises Stevia, aspartame, or a sugar alcohol, or a functional analog thereof, in an amount of about 5.5 wt. %. In specific embodiments, a composition comprises Stevia, aspartame, or a sugar alcohol, or a functional analog thereof, in an amount of about 6 wt. %. In specific embodiments, a composition comprises Stevia, aspartame, or a sugar alcohol, or a functional analog thereof, in an amount of about 6.5 wt. %.

In embodiments, a composition of the present disclosure comprises Stevia, aspartame, xylitol, or sorbitol, or a functional analog thereof, in an amount ranging from 0.5 wt. % to 60 wt. %. In some embodiments, a composition of the disclosure comprises Stevia, aspartame, xylitol, or sorbitol, or a functional analog thereof, in an amount ranging from 5 wt. % to 50 wt. %. In other embodiments, a composition of the disclosure comprises Stevia, aspartame, xylitol, or sorbitol, or a functional analog thereof, in an amount ranging from 10 wt. % to 40 wt. %. In further embodiments, a composition of the disclosure comprises Stevia, aspartame, xylitol, or sorbitol, or a functional analog thereof, in an amount ranging from 20 wt. % to 30 wt. %. In specific embodiments, a composition comprises Stevia, aspartame, xylitol, or sorbitol, or a functional analog thereof, in an amount of about 26 wt. %. In further embodiments, a composition of the disclosure comprises Stevia, aspartame, xylitol, or sorbitol, or a functional analog thereof, in an amount ranging from 2 wt. % to 10 wt. %. In specific embodiments, a composition comprises Stevia, aspartame, xylitol, or sorbitol, or a functional analog thereof, in an amount of about 2.5 wt. %. In specific embodiments, a composition comprises Stevia, aspartame, xylitol, or sorbitol, or a functional analog thereof, in an amount of about 3 wt. %. In specific embodiments, a composition comprises Stevia, aspartame, xylitol, or sorbitol, or a functional analog thereof, in an amount of about 3.5 wt. %. In specific embodiments, a composition comprises Stevia, aspartame, xylitol, or sorbitol, or a functional analog thereof, in an amount of about 4 wt. %. In specific embodiments, a composition comprises Stevia, aspartame, xylitol, or sorbitol, or a functional analog thereof, in an amount of about 4.5 wt. %. In specific embodiments, a composition comprises Stevia, aspartame, xylitol, or sorbitol, or a functional analog thereof, in an amount of about 5 wt. %. In specific embodiments, a composition comprises Stevia, aspartame, xylitol, or sorbitol, or a functional analog thereof, in an amount of about 5.5 wt. %. In specific embodiments, a composition comprises Stevia, aspartame, xylitol, or sorbitol, or a functional analog thereof, in an amount of about 6 wt. %. In specific embodiments, a composition comprises Stevia, aspartame, xylitol, or sorbitol, or a functional analog thereof, in an amount of about 6.5 wt. %.

In embodiments, a composition of the present disclosure comprises a sugar alcohol, or a functional analog thereof, in an amount ranging from 0.5 wt. % to 60 wt. %. In some embodiments, a composition of the disclosure comprises a sugar alcohol, or a functional analog thereof, in an amount ranging from 5 wt. % to 50 wt. %. In other embodiments, a composition of the disclosure comprises a sugar alcohol, or a functional analog thereof, in an amount ranging from 10 wt. % to 40 wt. %. In further embodiments, a composition of the disclosure comprises a sugar alcohol, or a functional analog thereof, in an amount ranging from 20 wt. % to 30 wt. %. In specific embodiments, a composition comprises a sugar alcohol, or a functional analog thereof, in an amount of about 26 wt. %. In further embodiments, a composition of the disclosure comprises a sugar alcohol, or a functional analog thereof, in an amount ranging from 2 wt. % to 10 wt. %. In specific embodiments, a composition comprises a sugar alcohol, or a functional analog thereof, in an amount of about 2.5 wt. %. In specific embodiments, a composition comprises a sugar alcohol, or a functional analog thereof, in an amount of about 3 wt. %. In specific embodiments, a composition comprises a sugar alcohol, or a functional analog thereof, in an amount of about 3.5 wt. %. In specific embodiments, a composition comprises a sugar alcohol, or a functional analog thereof, in an amount of about 4 wt. %. In specific embodiments, a composition comprises a sugar alcohol, or a functional analog thereof, in an amount of about 4.5 wt. %. In specific embodiments, a composition comprises a sugar alcohol, or a functional analog thereof, in an amount of about 5 wt. %. In specific embodiments, a composition comprises a sugar alcohol, or a functional analog thereof, in an amount of about 5.5 wt. %. In specific embodiments, a composition comprises a sugar alcohol, or a functional analog thereof, in an amount of about 6 wt. %. In specific embodiments, a composition comprises a sugar alcohol, or a functional analog thereof, in an amount of about 6.5 wt. %.

In embodiments, a composition of the present disclosure comprises xylitol, or a functional analog thereof, in an amount ranging from 0.5 wt. % to 60 wt. %. In some embodiments, a composition of the disclosure comprises xylitol, or a functional analog thereof, in an amount ranging from 5 wt. % to 50 wt. %. In other embodiments, a composition of the disclosure comprises xylitol, or a functional analog thereof, in an amount ranging from 10 wt. % to 40 wt. %. In further embodiments, a composition of the disclosure comprises xylitol, or a functional analog thereof, in an amount ranging from 20 wt. % to 30 wt. %. In specific embodiments, a composition comprises xylitol, or a functional analog thereof, in an amount of about 26 wt. %. In further embodiments, a composition of the disclosure comprises xylitol, or a functional analog thereof, in an amount ranging from 2 wt. % to 10 wt. %. In specific embodiments, a composition comprises xylitol, or a functional analog thereof, in an amount of about 2.5 wt. %. In specific embodiments, a composition comprises xylitol, or a functional analog thereof, in an amount of about 3 wt. %. In specific embodiments, a composition comprises xylitol, or a functional analog thereof, in an amount of about 3.5 wt. %. In specific embodiments, a composition comprises xylitol, or a functional analog thereof, in an amount of about 4 wt. %. In specific embodiments, a composition comprises xylitol, or a functional analog thereof, in an amount of about 4.5 wt. %. In specific embodiments, a composition comprises xylitol, or a functional analog thereof, in an amount of about 5 wt. %. In specific embodiments, a composition comprises xylitol, or a functional analog thereof, in an amount of about 5.5 wt. %. In specific embodiments, a composition comprises xylitol, or a functional analog thereof, in an amount of about 6 wt. %. In specific embodiments, a composition comprises xylitol, or a functional analog thereof, in an amount of about 6.5 wt. %.

In embodiments, a composition of the present disclosure comprises sorbitol in an amount ranging from 0.5 wt. % to 60 wt. %. In some embodiments, a composition of the disclosure comprises sorbitol in an amount ranging from 5 wt. % to 50 wt. %. In other embodiments, a composition of the disclosure comprises sorbitol in an amount ranging from 10 wt. % to 40 wt. %. In further embodiments, a composition of the disclosure comprises sorbitol in an amount ranging from 20 wt. % to 30 wt. %. In specific embodiments, a composition comprises sorbitol in an amount of about 26 wt. %. In further embodiments, a composition of the disclosure comprises sorbitol in an amount ranging from 2 wt. % to 10 wt. %. In specific embodiments, a composition comprises sorbitol in an amount of about 2.5 wt. %. In specific embodiments, a composition comprises sorbitol in an amount of about 3 wt. %. In specific embodiments, a composition comprises sorbitol in an amount of about 3.5 wt. %. In specific embodiments, a composition comprises sorbitol in an amount of about 4 wt. %. In specific embodiments, a composition comprises sorbitol in an amount of about 4.5 wt. %. In specific embodiments, a composition comprises sorbitol in an amount of about 5 wt. %. In specific embodiments, a composition comprises sorbitol in an amount of about 5.5 wt. %. In specific embodiments, a composition comprises sorbitol in an amount of about 6 wt. %. In specific embodiments, a composition comprises sorbitol in an amount of about 6.5 wt. %.

In various embodiments, a composition of the present disclosure comprises a sugar substitute in an amount ranging from 1 mg to 6 mg. In specific embodiments, a composition of the present disclosure comprises about 1 mg of a sugar substitute. In specific embodiments, a composition of the present disclosure comprises about 1.5 mg of a sugar substitute. In specific embodiments, a composition of the present disclosure comprises about 2 mg of a sugar substitute. In specific embodiments, a composition of the present disclosure comprises about 2.5 mg of a sugar substitute. In specific embodiments, a composition of the present disclosure comprises about 3 mg of a sugar substitute. In specific embodiments, a composition of the present disclosure comprises about 3.5 mg of a sugar substitute. In specific embodiments, a composition of the present disclosure comprises about 4 mg of a sugar substitute.

Embodiments of compositions of the disclosure comprise NaDCC, or a functional analog thereof, and a sugar substitute. In various embodiments, a composition of the present disclosure comprises: (A) NaDCC, or a functional analog thereof, in an amount ranging from 10 wt. % to 20 wt. %; and (B) a sugar substitute in an amount ranging from 2 wt. % to 10 wt. %. In some embodiments, a composition of the present disclosure comprises: (A) NaDCC, or a functional analog thereof, in an amount ranging from 13 wt. % to 17 wt. %; and (B) a sugar substitute in an amount ranging from 2.5 wt. % to 4.5 wt. %.

In various embodiments, a composition of the present disclosure comprises: (A) NaDCC, or a functional analog thereof, in an amount ranging from 15 wt. % to 60 wt. %; and (B) a sugar substitute in an amount ranging from 0.5 wt. % to 60 wt. %. In some embodiments, a composition of the present disclosure comprises: (A) NaDCC, or a functional analog thereof, in an amount ranging from 20 wt. % to 50 wt. %; and (B) a sugar substitute in an amount ranging from 5 wt. % to 50 wt. %. In some embodiments, a composition of the present disclosure comprises: (A) NaDCC, or a functional analog thereof, in an amount ranging from 20 wt. % to 50 wt. %; and (B) a sugar substitute in an amount ranging from 10 wt. % to 40 wt. %.

Embodiments of compositions of the disclosure comprise NaDCC, or a functional analog thereof, and Stevia, aspartame, xylitol, or sorbitol, or a functional analog thereof. In various embodiments, a composition of the present disclosure comprises: (A) NaDCC, or a functional analog thereof, in an amount ranging from 10 wt. % to 20 wt. %; and (B) Stevia, aspartame, xylitol, or sorbitol, or a functional analog thereof, in an amount ranging from 2 wt. % to 10 wt. %. In some embodiments, a composition of the present disclosure comprises: (A) NaDCC, or a functional analog thereof, in an amount ranging from 13 wt. % to 17 wt. %; and (B) Stevia, aspartame, xylitol, or sorbitol, or a functional analog thereof, in an amount ranging from 2.5 wt. % to 4.5 wt. %.

Embodiments of compositions of the disclosure comprise NaDCC, or a functional analog thereof, and xylitol or sorbitol, or a functional analog thereof. In various embodiments, a composition of the present disclosure comprises: (A) NaDCC, or a functional analog thereof, in an amount ranging from 10 wt. % to 20 wt. %; and (B) xylitol or sorbitol, or a functional analog thereof, in an amount ranging from 2 wt. % to 10 wt. %. In some embodiments, a composition of the present disclosure comprises: (A) NaDCC, or a functional analog thereof, in an amount ranging from 13 wt. % to 17 wt. %; and (B) xylitol or sorbitol, or a functional analog thereof, in an amount ranging from 2.5 wt. % to 4.5 wt. %.

Embodiments of compositions of the disclosure comprise NaDCC, or a functional analog thereof, and xylitol, or a functional analog thereof. In various embodiments, a composition of the present disclosure comprises: (A) NaDCC, or a functional analog thereof, in an amount ranging from 10 wt. % to 20 wt. %; and (B) xylitol, or a functional analog thereof, in an amount ranging from 2 wt. % to 10 wt. %. In some embodiments, a composition of the present disclosure comprises: (A) NaDCC, or a functional analog thereof, in an amount ranging from 13 wt. % to 17 wt. %; and (B) xylitol, or a functional analog thereof, in an amount ranging from 2.5 wt. % to 4.5 wt. %.

Other embodiments of compositions of the disclosure comprise NaDCC, or a functional analog thereof, and xylitol, or a functional analog thereof. In various embodiments, a composition of the present disclosure comprises: (A) NaDCC, or a functional analog thereof, in an amount ranging from 15 wt. % to 60 wt. %; and (B) xylitol, or a functional analog thereof, in an amount ranging from 0.5 wt. % to 60 wt. %. In some embodiments, a composition of the present disclosure comprises: (A) NaDCC, or a functional analog thereof, in an amount ranging from 20 wt. % to 50 wt. %; and (B) xylitol, or a functional analog thereof, in an amount ranging from 5 wt. % to 50 wt. %. In some embodiments, a composition of the present disclosure comprises: (A) NaDCC, or a functional analog thereof, in an amount ranging from 20 wt. % to 50 wt. %; and (B) xylitol, or a functional analog thereof, in an amount ranging from 10 wt. % to 40 wt. %.

In specific embodiments, a composition of the present disclosure comprises: (A) NaDCC, or a functional analog thereof, in an amount ranging from 20 wt. % to 50 wt. %; and (B) xylitol, or a functional analog thereof, in an amount ranging from 20 wt. % to 30 wt. %. In particular embodiments, a composition of the present disclosure comprises: (A) NaDCC, or a functional analog thereof, in an amount of about 40 wt. %; and (B) xylitol, or a functional analog thereof, in an amount of about 26 wt. %.

In particular embodiments, a composition of the present disclosure comprises (A) from 5.5 mg to 11 mg of NaDCC, or a functional analog thereof; and (B) 1 mg to 6 mg of a sugar substitute.

In particular embodiments, a composition of the present disclosure comprises (A) about 40 mg of NaDCC, or a functional analog thereof; and (B) about 26 mg of xylitol, or a functional analog thereof.

In embodiments, a composition of the present disclosure further comprises an alkali metal carbonate. Any suitable alkali metal carbonate may be used, such as calcium carbonate, calcium bicarbonate, sodium carbonate, sodium bicarbonate, or a combination thereof. In some embodiments, a composition of the disclosure comprises calcium carbonate, calcium bicarbonate, or both. In other embodiments, a composition of the disclosure comprises sodium carbonate, sodium bicarbonate, or both. In various embodiments, a composition of the disclosure comprises sodium carbonate and sodium bicarbonate. In some embodiments, the alkali metal carbonate is anhydrous (e.g., anhydrous sodium carbonate).

Accordingly, embodiments of the present disclosure include compositions comprising (A) NaDCC, or a functional analog thereof; (B) a sugar substitute; and (C) an alkali metal carbonate (e.g., calcium carbonate, calcium bicarbonate, sodium carbonate, sodium bicarbonate, or a combination thereof). Additional embodiments of the present disclosure include compositions comprising (A) NaDCC, or a functional analog thereof; (B) Stevia, aspartame, sorbitol, or xylitol, or a functional analog thereof, or a combination thereof; and (C) an alkali metal carbonate (e.g., calcium carbonate, calcium bicarbonate, sodium carbonate, sodium bicarbonate, or a combination thereof).

In various embodiments, a composition of the present disclosure comprises an alkali metal carbonate in an amount ranging from 50 wt. % to 65 wt. %.

In various embodiments, a composition of the present disclosure comprises an alkali metal carbonate in an amount ranging from 0.5 wt. % to 35 wt. %. In further embodiments, a composition of the present disclosure comprises an alkali metal carbonate in an amount ranging from 1 wt. % to 25 wt. %. In other embodiments, a composition of the present disclosure comprises an alkali metal carbonate in an amount ranging from 3 wt. % to 15 wt. %. In specific embodiments, a composition of the disclosure comprises about 6 wt. % of an alkali metal carbonate.

In some embodiments, a composition of the present disclosure comprises a first alkali metal carbonate and a second alkali metal carbonate. In embodiments, a composition of the present disclosure comprises a first alkali metal carbonate in an amount ranging from 35 wt. % to 45 wt. %. In other embodiments, a composition of the present disclosure comprises a second alkali metal carbonate in an amount ranging from 15 wt. % to 20 wt. %. In specific embodiments, a composition of the disclosure comprises about 37 wt. % of a first alkali metal carbonate. In specific embodiments, a composition of the disclosure comprises about 38 wt. % of a first alkali metal carbonate. In specific embodiments, a composition of the disclosure comprises about 39 wt. % of a first alkali metal carbonate. In specific embodiments, a composition of the disclosure comprises about 40 wt. % of a first alkali metal carbonate. In specific embodiments, a composition of the disclosure comprises about 41 wt. % of a first alkali metal carbonate. In specific embodiments, a composition of the disclosure comprises about 42 wt. % of a first alkali metal carbonate. In specific embodiments, a composition of the disclosure comprises about 43 wt. % of a first alkali metal carbonate. In specific embodiments, a composition of the disclosure comprises about 15.5 wt. % of a second alkali metal carbonate. In specific embodiments, a composition of the disclosure comprises about 16 wt. % of a second alkali metal carbonate. In specific embodiments, a composition of the disclosure comprises about 16.5 wt. % of a second alkali metal carbonate. In specific embodiments, a composition of the disclosure comprises about 17 wt. % of a second alkali metal carbonate. In specific embodiments, a composition of the disclosure comprises about 17.5 wt. % of a second alkali metal carbonate. In specific embodiments, a composition of the disclosure comprises about 18 wt. % of a second alkali metal carbonate. In specific embodiments, a composition of the disclosure comprises about 18.5 wt. % of a second alkali metal carbonate. In specific embodiments, a composition of the disclosure comprises about 19 wt. % of a second alkali metal carbonate. In specific embodiments, a composition of the disclosure comprises about 19.5 wt. % of a second alkali metal carbonate. In specific embodiments, a composition of the disclosure comprises about 20 wt. % of a second alkali metal carbonate.

In embodiments, a composition of the present disclosure further comprises an acceptable carrier, diluent, or excipient. As used herein “acceptable carrier, diluent, or excipient” includes any carrier, excipient, glidant, sweetening agent, diluent, preservative, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In various embodiments, the composition also comprises a colorant. In some embodiments, the colorant is a synthetic dye. In some embodiments, the colorant is not oil based. In particular embodiments, the colorant is tartrazine.

In additional embodiments, the composition further comprises a flavor enhancer. In particular embodiments, the flavor enhancer is glycine. In various embodiments, the composition does not contain glucose, fructose, galactose, sucrose, lactose, maltose, or any combination thereof.

In additional embodiments, the composition further comprises a lubricant. A lubricant generally functions to improve the flow. Further, in embodiments where a composition is formulated as a tablet, a lubricant may prevent sticking of the mixture during the tableting process. Any suitable lubricant can be used, for example, glycine, alanine, sodium benzoate, sodium acetate, fumaric acid, polyethylenglycols (PEG), or a combination thereof. In specific embodiments, the lubricant is glycine.

Accordingly, embodiments of the present disclosure include compositions comprising (A) NaDCC, or a functional analog (e.g., a polymorph, solvate, or tautomer) thereof (B) a sugar substitute (e.g., xylitol, or a functional analog thereof); and (C) glycine. In further embodiments, such a composition of the disclosure also includes (D) an alkali metal carbonate (e.g., calcium carbonate, calcium bicarbonate, sodium carbonate, sodium bicarbonate, or a combination thereof).

The compositions of the disclosure may be formulated in any suitable manner. In embodiments, a composition of the disclosure is formulated as a solid (e.g., a tablet, granule(s), a powder, etc.). In some embodiments, a composition of the disclosure is in the form of a tablet. In other embodiments, a composition of the disclosure is in the form of granule(s). In further embodiments, a composition of the disclosure is in the form of a powder. In specific embodiments, the granule(s) or powder is contained in a capsule. In other embodiments, a composition of the disclosure is formulated as a liquid (e.g., an aqueous solution).

In particular embodiments where the composition is formulated as a solid, the solid is effervescent. “Effervescence” refers to reaction of acids and bases in water that produces carbon dioxide. In some embodiments, a composition of the disclosure comprises an acid and a base that, when contacted with water, effervesce. In some such embodiments, the base is an alkali metal carbonate, as discussed further above. In some embodiments, the acid is an aliphatic carboxylic acid. In particular embodiments, the acid is adipic acid, fumaric acid, citric acid, tartaric acid, or a combination thereof. In particular embodiments, the acid is adipic acid, citric acid, tartaric acid, or a combination thereof. In specific embodiments, the acid is adipic acid. Adipic acid is advantageously non-hygroscopic, which helps preserve the integrity and stability of the finished formulation, and slows the effervescent reaction when added to water such that most of the chlorine liberated dissolves into the solution. In specific embodiments, the acid is citric acid. In specific embodiments, the acid is tartaric acid.

In embodiments, a composition of the present disclosure comprises an acid in an amount ranging from 20 wt. % to 25 wt. %. In specific embodiments, a composition of the present disclosure comprises an acid in an amount of about 22 wt. %. In specific embodiments, a composition of the present disclosure comprises an acid in an amount of about 23 wt. %. In specific embodiments, a composition of the present disclosure comprises an acid in an amount of about 24 wt. %.

Accordingly, embodiments of the present disclosure include compositions comprising (A) sodium dichloroisocyanurate (NaDCC), or a functional analog thereof (B) a sugar substitute; (C) an alkali metal carbonate; and (D) an acid.

Embodiments of the present disclosure also include compositions comprising (A) sodium dichloroisocyanurate (NaDCC), or a functional analog thereof (B) a sugar substitute selected from Stevia, aspartame, xylitol, or sorbitol, or a functional analog thereof, or a combination thereof; (C) an alkali metal carbonate selected from calcium carbonate, calcium bicarbonate, sodium carbonate, sodium bicarbonate, or a combination thereof; and (D) an acid selected from adipic acid, citric acid, tartaric acid, or a combination thereof.

Embodiments of the present disclosure further include compositions comprising (A) NaDCC, or a functional analog thereof; (B) an alkali metal carbonate (e.g., sodium carbonate, sodium bicarbonate, calcium carbonate, calcium bicarbonate, or a combination thereof); and (C) an acid (e.g., citric acid, adipic acid, or tartaric acid, or a combination thereof). Further embodiments of the present disclosure include compositions comprising (A) NaDCC, or a functional analog thereof; (B) a sugar substitute (e.g., xylitol, or a functional analog thereof); (C) an alkali metal carbonate (e.g., sodium carbonate, sodium bicarbonate, calcium carbonate, calcium bicarbonate, or a combination thereof); and (D) an acid (e.g., citric acid, adipic acid, or tartaric acid, or a combination thereof).

In embodiments, the composition is formulated as an effervescent tablet. In some embodiments, the composition is formulated as an effervescent powder, granule(s), or both.

In embodiments, a composition of the present disclosure is formulated as a tablet in an amount ranging from 50 mg to 60 mg. In particular embodiments, a composition of the present disclosure is formulated as a tablet in an amount of about 55 mg.

In various embodiments, a composition of the present disclosure is formulated as a tablet having a thickness ranging from 1.5 millimeters (mm) to 2.25 mm. In embodiments, the tablet has a thickness ranging from 1.75 mm to 1.9 mm.

In various embodiments, a composition of the present disclosure is formulated as a tablet having a diameter ranging from 4.5 mm to 5 mm. In embodiments, the tablet has a diameter ranging from 4.7 mm to 4.8 mm.

In various embodiments, a composition of the present disclosure is formulated as a tablet with a hardness ranging from 25 to 55 Newtons. In embodiments, a composition of the present disclosure is formulated as a tablet with a hardness ranging from 30 to 50 Newtons.

In some embodiments, a composition of the present disclosure further comprises magnesium sulfate, sodium benzoate, or both. In particular embodiments, a composition of the present disclosure further comprises magnesium sulfate. In particular embodiments, a composition of the present disclosure further comprises sodium benzoate. In particular embodiments, a composition of the present disclosure further comprises magnesium sulfate and sodium benzoate.

In various embodiments, a composition of the present disclosure has a pH of at least about 7 when dissolved in a volume of water ranging from 0.5 ounces to three ounces. In various embodiments, a composition of the present disclosure has a pH of at least about 7 when dissolved in a volume of water ranging from 0.5 ounces to two ounces. In further embodiments, a composition of the present disclosure has a pH of at least about 7 when dissolved in about one ounce of water. In some embodiments, a composition of the present disclosure has a pH ranging from 7 to 8 when dissolved in a volume of water ranging from 0.5 ounces to three ounces. In some embodiments, a composition of the present disclosure has a pH ranging from 7 to 8 when dissolved in a volume of water ranging from 0.5 ounces to two ounces. In some embodiments, a composition of the present disclosure has a pH ranging from 7 to 8 when dissolved in a volume of water of about one ounce. In some embodiments, a composition of the present disclosure has a pH ranging from 7 to 8 when dissolved in a volume of water of about two ounces. In some embodiments, a composition of the present disclosure has a pH ranging from 7.0 to 7.5 when dissolved in a volume of water of about one ounce. In some embodiments, a composition of the present disclosure has a pH ranging from 7.0 to 7.5 when dissolved in a volume of water of about two ounces. In specific embodiments, a composition of the present disclosure has a pH of about 8 when dissolved in a volume of water ranging from about 0.5 ounces to three ounces. In specific embodiments, a composition of the present disclosure has a pH of about 8 when dissolved in a volume of water ranging from 0.5 ounces to two ounces. In further embodiments, a composition of the present disclosure has a pH of about 8 when dissolved in about one ounce of water.

In embodiments, a composition of the present disclosure produces an aqueous solution comprising from 50 parts per million (ppm) to 400 ppm of free chlorine when dissolved in a volume of water ranging from 0.5 ounces to two ounces. In embodiments, a composition of the present disclosure produces an aqueous solution comprising at least about 100 ppm of free chlorine when dissolved in a volume of water ranging from 0.5 ounces to two ounces. In embodiments, a composition of the present disclosure produces an aqueous solution comprising about 200 ppm of free chlorine thereof, when dissolved in a volume of water ranging from 0.5 ounces to two ounces.

In embodiments, a composition of the present disclosure produces an aqueous solution comprising from 50 ppm to 400 ppm of free chlorine when dissolved in about one ounce of water. In embodiments, a composition of the present disclosure produces an aqueous solution comprising at least about 100 ppm of free chlorine when dissolved in about one ounce of water. In embodiments, a composition of the present disclosure produces an aqueous solution comprising about 200 ppm of free chlorine when dissolved in about one ounce of water.

Further embodiments of the present disclosure include a composition as described above, which is dissolved in water to form an aqueous solution. In some embodiments, the composition is formulated as an aqueous solution comprising water in a volume ranging from 0.5 ounces to three ounces. In some embodiments, the composition is formulated as an aqueous solution comprising water in a volume ranging from 0.5 ounces to two ounces. In other embodiments, the composition is formulated as an aqueous solution comprising about one ounce of water.

In various embodiments, a composition of the present disclosure is formulated as an aqueous solution comprising a volume of water ranging from 0.5 ounces to two ounces and has a pH of at least about 7. In some embodiments, a composition of the present disclosure is formulated as an aqueous solution comprising a volume of water ranging from 0.5 ounces to two ounces and has a pH ranging from 7 to 8. In specific embodiments, a composition of the present disclosure is formulated as an aqueous solution comprising a volume of water ranging from 0.5 ounces to two ounces and has a pH of about 8.

In some embodiments, a composition of the present disclosure is formulated as an aqueous solution comprising about one ounce of water and has a pH of at least about 7. In some embodiments, a composition of the present disclosure is formulated as an aqueous solution comprising a volume of water of about one ounce and has a pH ranging from 7 to 8. In further embodiments, a composition of the present disclosure is formulated as an aqueous solution comprising about one ounce of water and has a pH of about 8.

In embodiments, a composition of the present disclosure is formulated as an aqueous solution comprising from 50 ppm to 400 ppm of free chlorine in a volume of water ranging from 0.5 ounces to two ounces. In embodiments, a composition of the present disclosure is formulated as an aqueous solution comprising at least about 100 ppm of free chlorine in a volume of water ranging from 0.5 ounces to two ounces. In embodiments, a composition of the present disclosure is formulated as an aqueous solution comprising about 200 ppm of free chlorine in a volume of water ranging from 0.5 ounces to two ounces.

In embodiments, a composition of the present disclosure is formulated as an aqueous solution comprising from 50 ppm to 400 ppm of free chlorine in about one ounce of water. In embodiments, a composition of the present disclosure is formulated as an aqueous solution comprising at least about 100 ppm of free chlorine in about one ounce of water. In embodiments, a composition of the present disclosure is formulated as an aqueous solution comprising about 200 ppm of free chlorine in about one ounce of water.

In various embodiments, the disintegration time for a compound of the disclosure in two ounces of water is no more than three minutes, with no agitation. In various embodiments, the disintegration time for a compound of the disclosure in two ounces of water is no more than 2.5 minutes, with no agitation. In embodiments, the disintegration time for a compound of the disclosure in two ounces of water ranges from one minute to three minutes, with no agitation. In some embodiments, the disintegration time for a compound of the disclosure in two ounces of water ranges from two minutes to 2.5 minutes, with no agitation.

In various embodiments, the dissolution time for a compound of the disclosure in two ounces of water is no more than six minutes, with no agitation. In various embodiments, the dissolution time for a compound of the disclosure in two ounces of water is no more than five minutes, with no agitation. In various embodiments, the dissolution time for a compound of the disclosure in two ounces of water is no more than 4.5 minutes, with no agitation. In embodiments, the dissolution time for a compound of the disclosure in two ounces of water ranges from two minutes to six minutes, with no agitation. In some embodiments, the dissolution time for a compound of the disclosure in two ounces of water ranges from three minutes to five minutes, with no agitation.

Embodiments of the present disclosure further provide methods of manufacturing the compositions described herein. In embodiments, such a method of manufacturing comprises combining NaDCC, or a functional analog thereof, and a sugar substitute (e.g., xylitol, or a functional analog thereof), into a mixture; and homogenizing the mixture. In some embodiments, the method further comprises forming the mixture into a tablet. Any suitable tableting process can be used. In some such embodiments, forming the tablet comprises pressing the mixture.

### Kits

Kits comprising a composition as described herein are also provided. In some embodiments, a kit further comprises written instructions for using a composition of the present disclosure to improve oral health.

In various embodiments, the written instructions may include instructions regarding dosage, method of use, and the like. The written instructions can be in the form of printed instructions provided within the kit, or the written instructions can be printed on a portion of the container housing the kit. Written instructions may be in the form of a sheet, pamphlet, brochure, CD-ROM, or computer-readable device, or can provide directions to locate instructions at a remote location, such as a website. The written instructions may be in English and/or in a national or regional language.

In various embodiments, kits of the present disclosure further comprise a plurality of unit doses (e.g., tablets) presented in a pack or dispenser device. The pack may, for example, comprise metal or plastic foil, such as a blister pack.

Kits can further comprise one or more components to test, control, or adjust the pH of a solution comprising a composition of the disclosure. Such components may include, for example, a pH balancing material (e.g., a buffer solution) aliquots of water (e.g., distilled water), pH strips, and the like.

Variations in contents of any of the kits described herein can be made. In various embodiments, content of the kit is provided in a compact container.

### Methods of Use

The present disclosure further provides for various methods, including methods of using a composition as described herein. For example, compositions of the present disclosure can be used in methods of controlling bad breath or improving oral health in a subject. Such methods may comprise contacting at least a portion of an oral cavity of a subject with an effective amount of a composition of the present disclosure. Additional methods may comprise contacting at least a portion of an oral cavity of a subject with an effective amount of a composition comprising NaDCC, or a functional analog (e.g., a polymorph, solvate, or tautomer) thereof, and another active component. Still additional methods may comprise contacting at least a portion of an oral cavity of a subject with an effective amount of a composition comprising NaDCC, or a functional analog thereof, and an alkali metal carbonate (e.g., calcium carbonate, calcium bicarbonate, sodium carbonate, sodium bicarbonate, or a combination thereof). Such compositions may be provided in any suitable form and may comprise one or more of the components described above.

In further embodiments, a method of the present disclosure further comprises dissolving a solid form of the composition (e.g., a tablet, powder, granule(s), etc.) in a volume of water ranging from 0.5 ounces to three ounces. In some embodiments, the volume of water ranges from 0.5 ounces to two ounces. In particular embodiments, the volume of water is about one ounce. In particular embodiments, the volume of water is about two ounces. The resulting solution may then be used and would contact the oral cavity of the subject.

In particular embodiments, the solution comprising the composition contacts the subject's oral cavity for a time period ranging from 30 seconds to two minutes. In specific embodiments, the solution contacts the subject's oral cavity for about one minute. In specific embodiments, the solution contacts the subject's oral cavity for about two minutes. In various embodiments, the subject may swish (i.e., move the liquid from side to side and/or front to back in the oral cavity) the solution while it is in contact with their oral cavity. In specific embodiments, methods of the disclosure further comprise expelling the solution after it has contacted the oral cavity for a period of time.

In particular embodiments, the composition contacts the subject's oral cavity repeatedly over a time period of days, weeks, months, or years. The timing, frequency, dose amount, and dose concentration of treatment with the compositions and methods disclosed herein may be determined and adjusted based on the subject's health and condition and response to treatment.

For example, in one embodiment a subject may be treated using the methods disclosed herein on a daily, weekly or bi-weekly basis until the subject's oral health improves. In another embodiment, a subject may be treated using the methods disclosed herein on a daily, weekly or bi-weekly basis to facilitate maintenance of a subject's oral health. In still other embodiments, a subject may be treated more frequently at the beginning of a course of treatment, e.g., daily, and then less frequently, e.g., weekly or bi-weekly, as the subject's oral health improves or stabilizes.

In various embodiments, compositions of the present disclosure control bad breath in the subject.

In various embodiments, compositions of the present disclosure improve the oral health of a subject by treating periodontal disease. In some such embodiments, treating periodontal disease comprises preventing periodontal disease. In specific embodiments, the periodontal disease is caused by a pathogenic agent. In particular embodiments, the pathogenic agent is bacteria, viruses, yeasts, or a combination thereof. Exemplary pathogenic agents comprise Aggregatibactor actinomycetemcomitans, Prophyromonas gingivalis, Tannerella forsythia, Prevotella intermedia, Dialister pneumosintes, Fusobacterium species, Campylobacter rectus, Paevimonas micra, Eubacterium species, Beta-hemolytic streptococci, Staphylococci, Gram-negative enteric rods, and Candida species. In other embodiments, the periodontal disease is caused by a host immune response.

In further embodiments, compositions of the present disclosure improve the oral health of a subject by inhibiting the formation of dental caries. In additional embodiments, compositions of the present disclosure improve the oral health of a subject by reducing plaque accumulation, inflammation, or combination thereof.

In various embodiments, improving the oral health of a subject results in a statistically significant improvement in at least one category (e.g., pocket depths; width of keratinized tissue; gingival recession; attachment level; bleeding on probing; furcation status; presence of inflammation; presence, degree, and/or distribution of plaque/biofilm; presence, degree, and/or distribution of calculus; caries; proximal contact relationships endodontic/periodontal lesions; status of dental restorations and prosthetic appliances; other tooth or implant related problems; degree of mobility of teeth and dental implants; occlusal patterns; fremitus; quality/quantity of bone; and bone loss patterns) assessed in the Comprehensive Periodontal Evaluation established by the American Academy of Periodontology.

Various embodiments of the disclosure are described herein. It will be recognized that features specified in each embodiment may be combined with other specified features to provide further embodiments of the present disclosure. The following embodiments are included within the scope of the disclosure:

Embodiment 1. A composition, comprising:

sodium dichloroisocyanurate (NaDCC), or a functional analog thereof; and

a sugar substitute.

Embodiment 2. The composition of embodiment 1, wherein the NaDCC is present in an amount ranging from 10 weight percent (wt. %) to 20 wt. %.

Embodiment 3. The composition of embodiment 1 or 2, wherein the NaDCC is present in an amount ranging from 11 wt. % to 20 wt. %.

Embodiment 4. The composition of any one of embodiments 1-3, wherein the NaDCC is present in an amount of about 13 wt. %.

Embodiment 5. The composition of any one of embodiments 1-3, wherein the NaDCC is present in an amount of about 14 wt. %.

Embodiment 6. The composition of any one of embodiments 1-3, wherein the NaDCC is present in an amount of about 15 wt. %.

Embodiment 7. The composition of any one of embodiments 1-3, wherein the NaDCC is present in an amount of about 16 wt. %.

Embodiment 8. The composition of any one of embodiments 1-3, wherein the NaDCC is present in an amount of about 17 wt. %.

Embodiment 9. The composition of embodiment 1, wherein the NaDCC is present in an amount ranging from 15 wt. % to 60 wt. %.

Embodiment 10. The composition of embodiment 1, wherein the NaDCC is present in an amount ranging from 20 wt. % to 50 wt. %.

Embodiment 11. The composition of embodiment 9 or 10, wherein the NaDCC is present in an amount of about 40 wt. %.

Embodiment 12. The composition of any one of embodiments 1-4, wherein the composition comprises from 5 mg to 200 mg of the NaDCC, or the functional analog thereof.

Embodiment 13. The composition of any one of embodiments 1-12, wherein the NaDCC is present in an amount ranging from 5.5 milligrams (mg) to 11 mg.

Embodiment 14. The composition of any one of embodiments 1-13, wherein the NaDCC is present in an amount ranging from 7 mg to 10 mg.

Embodiment 15. The composition of any one of embodiments 1-14, wherein the NaDCC is present in an amount of about 7 mg.

Embodiment 16. The composition of any one of embodiments 1-14, wherein the NaDCC is present in an amount of about 7.5 mg.

Embodiment 17. The composition of any one of embodiments 1-14, wherein the NaDCC is present in an amount of about 8 mg.

Embodiment 18. The composition of any one of embodiments 1-14, wherein the NaDCC is present in an amount of about 8.5 mg.

Embodiment 19. The composition of any one of embodiments 1-14, wherein the NaDCC is present in an amount of about 9 mg.

Embodiment 20. The composition of any one of embodiments 1-14, wherein the NaDCC is present in an amount of about 9.5 mg.

Embodiment 21. The composition of any one of embodiments 1-14, wherein the NaDCC is present in an amount of about 10 mg.

Embodiment 22. The composition of any one of embodiments 1-12, wherein the NaDCC is present in an amount ranging from 10 mg to 150 mg.

Embodiment 23. The composition of any one of embodiments 1-12, wherein the NaDCC is present in an amount ranging from 20 mg to 100 mg.

Embodiment 24. The composition of any one of embodiments 1-12, wherein the NaDCC is present in an amount ranging from 30 mg to 75 mg.

Embodiment 25. The composition of any one of embodiments 1-12, wherein the NaDCC is present in an amount of about 40 mg of the NaDCC.

Embodiment 26. The composition of any one of embodiments 1-25, wherein the sugar substitute comprises Stevia, aspartame, or a sugar alcohol, or a functional analog thereof, or a combination thereof.

Embodiment 27. The composition of any one of embodiments 1-26, wherein the sugar substitute comprises Stevia, aspartame, or a sugar alcohol, or a functional analog thereof.

Embodiment 28. The composition of any one of embodiments 1-27, the sugar substitute comprises Stevia, aspartame, xylitol, or sorbitol, or a functional analog thereof, or a combination thereof.

Embodiment 29. The composition of any one of embodiments 1-28, the sugar substitute comprises Stevia, aspartame, xylitol, or sorbitol, or a functional analog thereof.

Embodiment 30. The composition of any one of embodiments 1-29, wherein the sugar substitute comprises Stevia, or a functional analog thereof.

Embodiment 31. The composition of any one of embodiments 1-30, wherein the sugar substitute comprises aspartame, or a functional analog thereof.

Embodiment 32. The composition of any one of embodiments 1-31, wherein the sugar substitute comprises xylitol, or a functional analog thereof.

Embodiment 33. The composition of any one of embodiments 1-32, wherein the sugar substitute comprises sorbitol, or a functional analog thereof.

Embodiment 34. The composition of any one of embodiments 1-33, wherein the sugar substitute is Stevia, aspartame, or a sugar alcohol, or a functional analog thereof.

Embodiment 35. The composition of any one of embodiments 1-34, the sugar substitute is Stevia, aspartame, xylitol, or sorbitol, or a functional analog thereof.

Embodiment 36. The composition of any one of embodiments 1-34, wherein the sugar substitute is Stevia, or a functional analog thereof.

Embodiment 37. The composition of any one of embodiments 1-34, wherein the sugar substitute is aspartame, or a functional analog thereof.

Embodiment 38. The composition of any one of embodiments 1-34, wherein the sugar substitute is xylitol, or a functional analog thereof.

Embodiment 39. The composition of any one of embodiments 1-34, wherein the sugar substitute is sorbitol, or a functional analog thereof.

Embodiment 40. The composition of any one of embodiments 1-34, wherein the sugar substitute is Stevia.

Embodiment 41. The composition of any one of embodiments 1-34, wherein the sugar substitute is aspartame.

Embodiment 42. The composition of any one of embodiments 1-34, wherein the sugar substitute is xylitol.

Embodiment 43. The composition of any one of embodiments 1-34, wherein the sugar substitute is sorbitol.

Embodiment 44. The composition of any one of embodiments 1-43, wherein the sugar substitute is present in an amount ranging from 0.5 wt. % to 60 wt. %.

Embodiment 45. The composition of any one of embodiments 1-44, wherein the sugar substitute is present in an amount ranging from 5 wt. % to 50 wt. %.

Embodiment 46. The composition of any one of embodiments 1-44, wherein the sugar substitute is present in an amount ranging from 2 wt. % to 10 wt. %.

Embodiment 47. The composition of any one of embodiments 1-44, wherein the sugar substitute is present in an amount of about 2.5 wt. %.

Embodiment 48. The composition of any one of embodiments 1-44, wherein the sugar substitute is present in an amount of about 3 wt. %.

Embodiment 49. The composition of any one of embodiments 1-44, wherein the sugar substitute is present in an amount of about 3.5 wt. %.

Embodiment 50. The composition of any one of embodiments 1-44, wherein the sugar substitute is present in an amount of about 4 wt. %.

Embodiment 51. The composition of any one of embodiments 1-44, wherein the sugar substitute is present in an amount of about 4.5 wt. %.

Embodiment 52. The composition of any one of embodiments 1-44, wherein the sugar substitute is present in an amount of about 5 wt. %.

Embodiment 53. The composition of any one of embodiments 1-44, wherein the sugar substitute is present in an amount of about 5.5 wt. %.

Embodiment 54. The composition of any one of embodiments 1-44, wherein the sugar substitute is present in an amount of about 6 wt. %.

Embodiment 55. The composition of any one of embodiments 1-44, wherein the sugar substitute is present in an amount of about 6.5 wt. %.

Embodiment 56. The composition of any one of embodiments 1-55, wherein the sugar substitute is present in an amount ranging from 1 mg to 6 mg.

Embodiment 57. The composition of any one of embodiments 1-56, wherein the sugar substitute is present in an amount of about 1 mg.

Embodiment 58. The composition of any one of embodiments 1-56, wherein the sugar substitute is present in an amount of about 1.5 mg.

Embodiment 59. The composition of any one of embodiments 1-56, wherein the sugar substitute is present in an amount of about 2 mg.

Embodiment 60. The composition of any one of embodiments 1-56, wherein the sugar substitute is present in an amount of about 2.5 mg.

Embodiment 61. The composition of any one of embodiments 1-56, wherein the sugar substitute is present in an amount of about 3 mg.

Embodiment 62. The composition of any one of embodiments 1-56, wherein the sugar substitute is present in an amount of about 3.5 mg.

Embodiment 63. The composition of any one of embodiments 1-56, wherein the sugar substitute is present in an amount of about 4 mg.

Embodiment 64. The composition of any one of embodiments 1-45, wherein the sugar substitute is present in an amount ranging from 10 wt. % to 40 wt. %.

Embodiment 65. The composition of any one of embodiments 1-45, wherein the sugar substitute is present in an amount ranging from 20 wt. % to 30 wt. %.

Embodiment 66. The composition of any one of embodiments 1-45, wherein the sugar substitute is present in an amount of about 26 wt. %.

Embodiment 67. The composition of any one of embodiments 1-66, further comprising an alkali metal carbonate.

Embodiment 68. The composition of embodiment 67, wherein the alkali metal carbonate comprises calcium carbonate, calcium bicarbonate, sodium carbonate, sodium bicarbonate, or a combination thereof.

Embodiment 69. The composition of embodiment 67 or 68, wherein the alkali metal carbonate is calcium carbonate, calcium bicarbonate, sodium carbonate, sodium bicarbonate, or a combination thereof.

Embodiment 70. The composition of any one of embodiments 67-69, wherein the alkali metal carbonate is calcium carbonate, calcium bicarbonate, or both.

Embodiment 71. The composition of any one of embodiments 67-69, wherein the alkali metal carbonate is sodium carbonate, sodium bicarbonate, or both.

Embodiment 72. The composition of any one of embodiments 67-71, wherein the alkali metal carbonate is present in an amount ranging from 50 wt. % to 65 wt. %.

Embodiment 73. The composition of any one of embodiments 67-71, wherein the alkali metal carbonate is present in an amount ranging from 0.5 wt. % to 35 wt. %.

Embodiment 74. The composition of any one of embodiments 67-71, wherein the alkali metal carbonate is present in an amount ranging from about 1 wt. % to 25 wt. %.

Embodiment 75. The composition of any one of embodiments 67-71, wherein the alkali metal carbonate is present in an amount ranging from 3 wt. % to 15 wt. %.

Embodiment 76. The composition of any one of embodiments 67-71, wherein the alkali metal carbonate is present in an amount of about 6 wt. %.

Embodiment 77. The composition of any one of embodiments 67-76, wherein the composition comprises a first alkali metal carbonate and a second alkali metal carbonate.

Embodiment 78. The composition of embodiment 77, wherein the first alkali metal carbonate in an amount ranging from 35 wt. % to 45 wt. %.

Embodiment 79. The composition of embodiment 77 or 78, wherein the first alkali metal carbonate in an amount of about 37 wt. %.

Embodiment 80. The composition of embodiment 77 or 78, wherein the first alkali metal carbonate in an amount of about 38 wt. %.

Embodiment 81. The composition of embodiment 77 or 78, wherein the first alkali metal carbonate in an amount of about 39 wt. %.

Embodiment 82. The composition of embodiment 77 or 78, wherein the first alkali metal carbonate in an amount of about 40 wt. %.

Embodiment 83. The composition of embodiment 77 or 78, wherein the first alkali metal carbonate in an amount of about 41 wt. %.

Embodiment 84. The composition of embodiment 77 or 78, wherein the first alkali metal carbonate in an amount of about 42 wt. %.

Embodiment 85. The composition of embodiment 77 or 78, wherein the first alkali metal carbonate in an amount of about 43 wt. %.

Embodiment 86. The composition of any one of embodiments 77-85, wherein the second alkali metal carbonate in an amount ranging from 15 wt. % to 20 wt. %.

Embodiment 87. The composition of any one of embodiments 77-86, wherein the second alkali metal carbonate in an amount of about 15.5 wt. %.

Embodiment 88. The composition of any one of embodiments 77-86, wherein the second alkali metal carbonate in an amount of about 16 wt. %.

Embodiment 89. The composition of any one of embodiments 77-86, wherein the second alkali metal carbonate in an amount of about 16.5 wt. %.

Embodiment 90. The composition of any one of embodiments 77-86, wherein the second alkali metal carbonate in an amount of about 17 wt. %.

Embodiment 91. The composition of any one of embodiments 77-86, wherein the second alkali metal carbonate in an amount of about 17.5 wt. %.

Embodiment 92. The composition of any one of embodiments 77-86, wherein the second alkali metal carbonate in an amount of about 18 wt. %.

Embodiment 93. The composition of any one of embodiments 77-86, wherein the second alkali metal carbonate in an amount of about 18.5 wt. %.

Embodiment 94. The composition of any one of embodiments 77-86, wherein the second alkali metal carbonate in an amount of about 19 wt. %.

Embodiment 95. The composition of any one of embodiments 77-86, wherein the second alkali metal carbonate in an amount of about 19.5 wt. %.

Embodiment 96. The composition of any one of embodiments 1-95, further comprising an acid.

Embodiment 97. The composition of embodiment 96, wherein the acid comprises adipic acid, citric acid, tartaric acid, or a combination thereof.

Embodiment 98. The composition of embodiment 96 or 97, wherein the acid is adipic acid, citric acid, tartaric acid, or a combination thereof.

Embodiment 99. The composition of any one of embodiments 96-98, wherein the acid is adipic acid.

Embodiment 100. The composition of any one of embodiments 96-98, wherein the acid is citric acid.

Embodiment 101. The composition of any one of embodiments 96-98, wherein the acid is tartaric acid.

Embodiment 102. The composition of any one of embodiments 96-101, wherein the acid is present in an amount ranging from 20 wt. % to 25 wt. %.

Embodiment 103. The composition of any one of embodiments 96-102, wherein the acid is present in an amount of about 22 wt. %.

Embodiment 104. The composition of any one of embodiments 96-102, wherein the acid is present in an amount of about 23 wt. %.

Embodiment 105. The composition of any one of embodiments 96-102, wherein the acid is present in an amount of about 24 wt. %.

Embodiment 106. The composition of any one of embodiments 1-105, further comprising magnesium sulfate, sodium benzoate, or both

Embodiment 107. The composition of any one of embodiments 1-105, further comprising magnesium sulfate.

Embodiment 108. The composition of any one of embodiments 1-105, further comprising sodium benzoate.

Embodiment 109. The composition of any one of embodiments 1-108, wherein the composition is a solid.

Embodiment 110. The composition of embodiment 109, wherein the solid is a tablet, a granule, or a powder.

Embodiment 111. The composition of any one of embodiments 1-109, wherein the composition is in the form of a tablet.

Embodiment 112. The composition of embodiment 110 or 111, wherein the tablet is effervescent.

Embodiment 113. The composition of embodiment 110, wherein the granule or the powder is in a capsule.

Embodiment 114. The composition of any one of embodiments 1-113, wherein the pH of the composition is at least 7 when dissolved in 0.5 to 3 ounces of water.

Embodiment 115. The composition of any one of embodiments 1-114, wherein the pH of the composition is at least 7 when dissolved in 0.5 to 2 ounces of water.

Embodiment 116. The composition of any one of embodiments 1-115, wherein the pH of the composition is at least 7 when dissolved in about 1 ounce of water.

Embodiment 117. The composition of any one of embodiments 1-116, wherein the pH of the composition is at least 7 when dissolved in about 2 ounces of water.

Embodiment 118. The composition of any one of embodiments 1-117, wherein the pH of the composition ranges from 7 to 8 when dissolved in 0.5 to 3 ounces of water.

Embodiment 119. The composition of any one of embodiments 1-118, wherein the pH of the composition ranges from 7 to 8 when dissolved in 0.5 to 2 ounces of water.

Embodiment 120. The composition of any one of embodiments 1-119, wherein the pH of the composition ranges from 7 to 8 when dissolved in about 1 ounce of water.

Embodiment 121. The composition of any one of embodiments 1-120, wherein the pH of the composition ranges from 7 to 8 when dissolved in about 2 ounces of water.

Embodiment 122. The composition of any one of embodiments 1-108, wherein the composition is a solution comprising 0.5 to 3 ounces of water.

Embodiment 123. The composition of any one of embodiments 1-108, wherein the composition is a solution comprising 0.5 to 2 ounces of water.

Embodiment 124. The composition of embodiment 122 or 123, wherein the solution comprises about 1 ounce of water.

Embodiment 125. The composition of embodiment 122 or 123, wherein the solution comprises about 2 ounces of water.

Embodiment 126. The composition of any one of embodiments 122-125, wherein the solution comprises from about 50 parts per million (ppm) to 400 ppm of free chlorine.

Embodiment 127. The composition of any one of embodiments 122-126, wherein the solution comprises at least 100 ppm of free chlorine.

Embodiment 128. The composition of any one of embodiments 122-127, wherein the solution comprises about 200 ppm of free chlorine.

Embodiment 129. The composition of any one of embodiments 122-128, wherein the pH of the solution is at least 7.

Embodiment 130. The composition of any one of embodiments 122-129, wherein the pH of the solution ranges from 7 to 8.

Embodiment 131. The composition of any one of embodiments 1-130, further comprising tartrazine.

Embodiment 132. The composition of any one of embodiments 1-131, further comprising glycine.

Embodiment 133. The composition of any one of embodiments 1-132, wherein the composition does not contain glucose, fructose, galactose, sucrose, lactose, maltose, or a combination thereof.

Embodiment 134. A method of manufacturing a composition of any one of embodiments 1-133, the method comprising:

combining sodium dichloroisocyanurate (NaDCC), or the functional analog thereof, and the sugar substitute into a mixture; and

homogenizing the mixture.

Embodiment 135. The method of embodiment 134, further comprising forming the mixture into a tablet.

Embodiment 136. The method of embodiments 134 or 135, wherein the forming the tablet comprises pressing the mixture.

Embodiment 137. A method of controlling bad breath in a subject, the method comprising:

contacting at least a portion of an oral cavity of the subject with an effective amount a composition of any one of embodiments 1-133.

Embodiment 138. A method of improving oral health in a subject, the method comprising:

contacting at least a portion of an oral cavity of the subject with an effective amount a composition of any one of embodiments 1-133.

Embodiment 139. A method of improving oral health in a subject, the method comprising:

contacting at least a portion of an oral cavity of the subject with an effective amount a composition comprising sodium dichloroisocyanurate (NaDCC), or a functional analog thereof, and an alkali metal carbonate.

Embodiment 140. The method of embodiment 139, wherein the composition comprises from 15 wt. % to 60 wt. % of the NaDCC, or the functional analog thereof.

Embodiment 141. The method of embodiments 139 or 140, wherein the composition comprises from 20 wt. % to 50 wt. % of the NaDCC, or the functional analog thereof.

Embodiment 142. The composition of any one of embodiments 139-141, wherein the composition comprises about 40 wt. % of the NaDCC, or the functional analog thereof.

Embodiment 143. The composition of any one of embodiments 139-142, wherein the composition comprises from 5 mg to 200 mg of the NaDCC, or the functional analog thereof.

Embodiment 144. The method of any one of embodiments 139-143, wherein the composition comprises from 10 mg to 150 mg of the NaDCC, or the functional analog thereof.

Embodiment 145. The method of any one of embodiments 139-144, wherein the composition comprises from 20 mg to 100 mg of the NaDCC, or the functional analog thereof.

Embodiment 146. The method of any one of embodiments 139-145, wherein the composition comprises from 30 mg to 75 mg of the NaDCC, or the functional analog thereof.

Embodiment 147. The method of any one of embodiments 139-146, wherein the composition comprises about 40 mg of the NaDCC, or the functional analog thereof.

Embodiment 148. The method of any one of embodiments 139-147, wherein the composition comprises from 0.5 wt. % to 35 wt. % of the alkali metal carbonate.

Embodiment 149. The method of any one of embodiments 139-148, wherein the composition comprises from 1 wt. % to 25 wt. % of the alkali metal carbonate.

Embodiment 150. The method of any one of embodiments 139-149, wherein the composition comprises from 3 wt. % to 15 wt. % of the alkali metal carbonate.

Embodiment 151. The method of any one of embodiments 139-150, wherein the composition comprises about 6 wt. % of the alkali metal carbonate.

Embodiment 152. The method of any one of embodiments 139-151, wherein the alkali metal carbonate is calcium bicarbonate, calcium carbonate, or both.

Embodiment 153. The method of any one of embodiments 139-152, further comprising an acid.

Embodiment 154. The method of embodiment 153, wherein the acid is adipic acid.

Embodiment 155. The method of any one of embodiments 139-154, wherein the composition comprises xylitol, or a functional analog thereof.

Embodiment 156. The method of embodiment 155, wherein the composition comprises from 0.5 wt. % to 60 wt. % of the xylitol, or the functional analog thereof.

Embodiment 157. The method of embodiment 155 or 156, wherein the composition comprises from 5 wt. % to 50 wt. % of the xylitol, or the functional analog thereof.

Embodiment 158. The method of any one of embodiments 155-157, wherein the composition comprises from 10 wt. % to 40 wt. % of the xylitol, or the functional analog thereof.

Embodiment 159. The method of any one of embodiments 155-158, wherein the composition comprises from 20 wt. % to 30 wt. % of the xylitol, or the functional analog thereof.

Embodiment 160. The method of any one of embodiments 155-159, wherein the composition comprises about 26 wt. % of the xylitol, or the functional analog thereof.

Embodiment 161. The method of any one of embodiments 139-160, further comprising tartrazine.

Embodiment 162. The method of any one of embodiments 139-161, further comprising glycine.

Embodiment 163. The method of any one of embodiments 139-162, wherein the composition does not contain glucose, fructose, galactose, sucrose, lactose, maltose, or a combination thereof.

Embodiment 164. The method of any one of embodiments 139-163, wherein the composition is a solid.

Embodiment 165. The method of embodiment 164, wherein the solid is a tablet, a granule, or a powder.

Embodiment 166. The method of any one of embodiments 139-165, wherein the composition is in the form of a tablet.

Embodiment 167. The method of embodiment 165 or 166, wherein the tablet is effervescent.

Embodiment 168. The method of embodiment 165, wherein the granule or the powder is in a capsule.

Embodiment 169. The method of any one of embodiments 137-168, further comprising preparing a solution by dissolving the composition in from 0.5 to 3 ounces of water, wherein the solution contacts the at least the portion of the oral cavity.

Embodiment 170. The method of any one of embodiments 137-169, further comprising preparing a solution by dissolving the composition in from 0.5 to 2 ounces of water, wherein the solution contacts the at least the portion of the oral cavity.

Embodiment 171. The method of any one of embodiments 137-170, further comprising preparing a solution by dissolving the composition in about 1 ounce of water, wherein the solution contacts the at least the portion of the oral cavity.

Embodiment 172. The method of any one of embodiments 137-170, further comprising preparing a solution by dissolving the composition in about 2 ounces of water, wherein the solution contacts the at least the portion of the oral cavity.

Embodiment 173. The method of any one of embodiments 169-172, wherein the pH of the solution is at least 7.

Embodiment 174. The method of any one of embodiments 169-173, wherein the pH of the solution ranges from 7 to 8.

Embodiment 175. The method of any one of embodiments 137-174, further comprising preparing a solution by dissolving the composition in about 1 ounce of water, wherein the solution contacts the at least the portion of the oral cavity.

Embodiment 176. The method of any one of embodiments 137-174, further comprising preparing a solution by dissolving the composition in about 2 ounces of water, wherein the solution contacts the at least the portion of the oral cavity.

Embodiment 177. The method of any one of embodiments 169-176, wherein the solution comprises from about 50 ppm to 400 ppm of free chlorine.

Embodiment 178. The method of any one of embodiments 169-177, wherein the solution comprises about 200 ppm of free chlorine.

Embodiment 179. The method of any one of embodiments 169-178, wherein the solution contacts the at least the portion of the oral cavity for 0.5 to 2 minutes.

Embodiment 180. The method of any one of embodiments 169-179, wherein the solution contact at least the portion of the oral cavity for about 1 minute.

Embodiment 181. The method of any one of embodiments 138-180, wherein improving oral health comprises reducing plaque accumulation, inflammation, or combination thereof.

Embodiment 182. The method of any one of embodiments 138-181, wherein improving oral health comprises preventing periodontal disease.

Embodiment 183. The method of embodiment 182, wherein the periodontal disease is caused by a pathogenic agent.

Embodiment 184. The method of embodiment 183, wherein the pathogenic agent is bacteria, viruses, yeasts, or a combination thereof.

Embodiment 185. The method of any one of embodiments 182-184, wherein the periodontal disease is caused by a host immune response.

Embodiment 186. The method of any one of embodiments 138-185, wherein improving oral health comprises inhibiting the formation of dental caries.

Embodiment 187. The method of any one of embodiments 138-186, further comprising expelling the composition after the contacting.

Embodiments of this disclosure are further illustrated by the following examples.

Embodiments of the present disclosure include a composition, comprising: NaDCC, or a functional analog thereof; a sugar substitute selected from Stevia, aspartame, xylitol, or sorbitol, or a functional analog thereof, or a combination thereof; an alkali metal carbonate selected from calcium carbonate, calcium bicarbonate, sodium carbonate, sodium bicarbonate, or a combination thereof; and an acid selected from adipic acid, citric acid, tartaric acid, or a combination thereof.

In some embodiments, the NaDCC is present in an amount ranging from 10 wt. % to 20 wt. %. In some embodiments, the sugar substitute is present in an amount ranging from 2 wt. % to 10 wt. %. In some embodiments, the alkali metal carbonate is present in an amount ranging from 50 wt. % to 65 wt. %. In some embodiments, the acid is present in an amount ranging from 20 wt. % to 25 wt. %.

Accordingly, embodiments of the present disclosure comprise a composition, comprising: NaDCC, or a functional analog thereof in an amount ranging from 10 wt. % to 20 wt. %; a sugar substitute selected from Stevia, aspartame, xylitol, or sorbitol, or a functional analog thereof, or a combination thereof, in an amount ranging from 2 wt. % to 10 wt. %; an alkali metal carbonate selected from calcium carbonate, calcium bicarbonate, sodium carbonate, sodium bicarbonate, or a combination thereof, in an amount ranging from 50 wt. % to 65 wt. %; and an acid selected from adipic acid, citric acid, tartaric acid, or a combination thereof in an amount ranging from 20 wt. % to 25 wt. %.

In embodiments, the alkali metal carbonate is sodium carbonate, sodium bicarbonate, or both. In various embodiments, the composition comprises a first alkali metal carbonate and a second alkali metal carbonate. In some embodiments, the first alkali metal carbonate in an amount ranging from 35 wt. % to 45 wt. %. In some embodiments, the second alkali metal carbonate in an amount ranging from 15 wt. % to 20 wt. %. In various embodiments, the composition further comprises magnesium sulfate. In various embodiments, the composition further comprises sodium benzoate.

In various embodiments, the composition is in the form of a tablet. In some embodiments, the tablet is effervescent. In other embodiments, the composition is in the form of a granule or powder, and wherein the granule or powder is in a capsule. In some embodiments, the pH of the composition is at least 7 when dissolved in 0.5 to 3 ounces of water. In some embodiments, the pH of the composition ranges from 7 to 8 when dissolved in 0.5 to 3 ounces of water.

In embodiments, the composition is a solution comprising 0.5 to 3 ounces of water. In some embodiments, the solution comprises at least 100 ppm of free chlorine. In some embodiments, the pH of the solution is at least 7. In some embodiments, the pH of the solution ranges from 7 to 8.

Embodiments of the present disclosure further include a method of manufacturing a composition of the present disclosure, the method comprising: combining the NaDCC, or the functional analog thereof, the sugar substitute, the alkali metal carbonate, and the acid into a mixture; and homogenizing the mixture.

Embodiments of the present disclosure further include a method of controlling bad breath in a subject, the method comprising: contacting at least a portion of an oral cavity of the subject with an effective amount a composition of the present disclosure. In various embodiments, the solution contacts the at least the portion of the oral cavity for 0.5 to 2 minutes. In further embodiments, the method further comprises expelling the composition after the contacting.

### EXAMPLES

### Example 1

### Formulations Tested

The compositions of representative samples used throughout these Examples are provided in Table 1.

TABLE 1

| | NaDCC | Adipic | Sodium | Sodium | Xylitol - | Xylitol - |
|---|---|---|---|---|---|---|
| | anhydrous | acid | bicarbonate | carbonate | Sample 1 | Sample 2 |
| LB0148-5-07 | 17.00% | 22.00% | 37.00% | 24.00% | 0.00% | 0.00% |
| LB0148-5-07-1 | | | | | | |
| LB0148-5-07-GX2 | | | | | | |
| LB0148-15-01 | 17.00% | 22.00% | 36.50% | 23.50% | 1.00% | 0.00% |
| LB0148-15-02 | 17.00% | 22.00% | 36.00% | 23.00% | 2.00% | 0.00% |
| LB0148-15-03 | 17.00% | 21.00% | 36.00% | 23.00% | 3.00% | 0.00% |
| LB0148-15-04 | 17.00% | 21.00% | 35.00% | 23.00% | 4.00% | 0.00% |
| LB0148-15-05 | 17.00% | 21.00% | 34.00% | 23.00% | 5.00% | 0.00% |
| LB0148-15-01-1 | 17.00% | 22.00% | 36.50% | 23.50% | 0.00% | 1.00% |
| LB0148-15-01-GX2 | 17.00% | 22.00% | 36.50% | 23.50% | 0.00% | 1%* |
| LB0148-15-02-1 | 17.00% | 22.00% | 36.00% | 23.00% | 0.00% | 2.00% |
| LB0148-15-02-GX2 | 17.00% | 22.00% | 36.00% | 23.00% | 0.00% | 2%* |
| LB0148-15-03-1 | 17.00% | 21.00% | 36.00% | 23.00% | 0.00% | 3.00% |
| LB0148-15-03-GX2 | 17.00% | 21.00% | 36.00% | 23.00% | 0.00% | 3%* |
| LB0148-15-04-1 | 17.00% | 21.00% | 35.00% | 23.00% | 0.00% | 4.00% |
| LB0148-15-04-GX2 | 17.00% | 21.00% | 35.00% | 23.00% | 0.00% | 4%* |
| LB0148-15-05-1 | 17.00% | 21.00% | 34.00% | 23.00% | 0.00% | 5.00% |
| LB0148-15-05-GX2 | 17.00% | 21.00% | 34.00% | 23.00% | 0.00% | 5%* |
| LB0148-24-01 | 15.16% | 29.66% | 41.88% | 13.30% | 0.00% | 0.00% |
| 917 mg | | | | | | |
| LB0148-24-02 | 15.16% | 21.46% | 34.77% | 23.61% | 5.00% | 0.00% |
| 917 mg | | | | | | |
| LB0148-24-03 | 15.16% | 22.46% | 34.77% | 22.61% | 5.00% | 0.00% |
| 917 mg | | | | | | |
| LB0148-24-04 | 15.16% | 23.46% | 35.77% | 20.61% | 5.00% | 0.00% |
| 917 mg | | | | | | |
| LB0148-24-05 | 15.16% | 24.46% | 36.77% | 18.61% | 5.00% | 0.00% |
| 917 mg | | | | | | |
| LB0148-24-06 | 15.16% | 22.46% | 35.77% | 21.61% | 5.00% | 0.00% |
| 917 mg | | | | | | |
| LB0148-24-07 | 15.16% | 23.46% | 34.77% | 21.61% | 5.00% | 0.00% |
| 917 mg | | | | | | |
| LB0148-24-08 | 15.16% | 24.46% | 34.77% | 20.61% | 5.00% | 0.00% |
| 917 mg | | | | | | |
| LB0148-24-09 | 9.81% | 31.52% | 44.53% | 14.14% | 0.00% | 0.00% |
| 1417 mg | | | | | | |
| LB0148-24-10 | 9.81% | 22.90% | 37.10% | 25.19% | 5.00% | 0.00% |
| 1417 mg | | | | | | |
| LB0148-24-11 | 9.81% | 23.90% | 37.10% | 24.19% | 5.00% | 0.00% |
| 1417 mg | | | | | | |
| LB0148-24-12 | 9.81% | 24.90% | 38.10% | 22.19% | 5.00% | 0.00% |
| 1417 mg | | | | | | |
| LB0148-24-13 | 9.81% | 25.90% | 39.10% | 20.19% | 5.00% | 0.00% |
| 1417 mg | | | | | | |
| LB0148-24-14 | 9.81% | 23.90% | 38.10% | 23.19% | 5.00% | 0.00% |
| 1417 mg | | | | | | |
| LB0148-24-15 | 9.81% | 24.90% | 37.10% | 23.19% | 5.00% | 0.00% |
| 1417 mg | | | | | | |
| LB0148-24-16 | 9.81% | 25.90% | 37.10% | 22.19% | 5.00% | 0.00% |
| 1417 mg | | | | | | |

*Xylitol was ground.

### Baseline—pH and Chlorine Content of Dissolved Compositions

Control formulations was manufactured and tested to establish a baseline. The Control 1 (3-03-0128) formulation contained 8.5 mg NaDCC with a 49 mg finished weight. The Control 2 formulation had a total weight of 817 mg. Control 2 was (1) dissolved in 1000 milliliters (mL) of deionized (DI) water, and (2) compressed into a tablet and dissolved 1000 mL of DI water, both of which had a resulting pH of less than 6.5. The difference in the resulting pH between the Control 2 powder and Control 2 tablet was no more than 0.14%.

When tested for chlorine content, the free chlorine was less than 100 ppm in all replicates. However, some results were less than 80 ppm. When comparing the Control 2 formulation powder and the Control 2 tablet, the difference in free chlorine observed for each replicate was 12.16%, 5.48%, and 10.45%. These results indicate that the formulation being compressed impacts the resulting chlorine content.

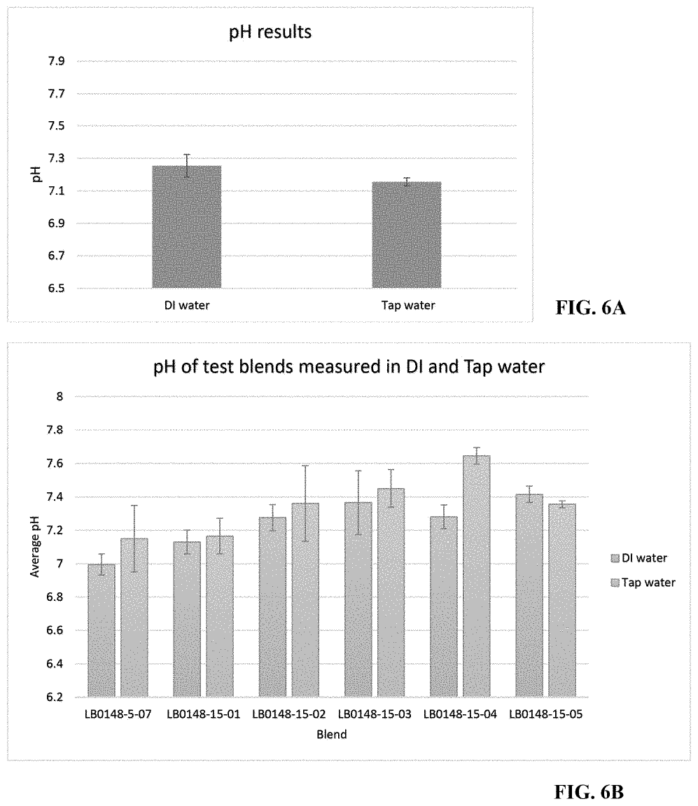

Effervescent components, adipic acid, sodium bicarbonate and sodium carbonate were adjusted in order to increase the pH to between 7-8. Seven formulations were tested in total. A control formulation was chosen (NaDCC: 17%; Adipic acid: 22%; Sodium bicarbonate: 37%; and Sodium carbonate: 24%, “Control 3” (LB0148-5-07) due to the pH of the resulting solution being 7.08 when dissolved in DI water and 7.04 when dissolved in tap water (see, FIG. 6A).

### Xylitol Interaction Studies

A series of xylitol:NaDCC ratios were examined ranging from 1:12 to 1:1. No greater than a 1.59% change in chlorine content was observed within this range. These results indicate that the chlorine content was not affected by the addition of xylitol.

Next, the impact of incorporating xylitol on pH was investigated. Five test blends were dissolved in DI and tap water and compared to a control formulation (Control 3 (LB0148-5-07)). The five test blends, Compositions 1-5 (LB0148-15-01, LB0148-15-02, LB0148-15-03, LB0148-15-04, LB0148-15-05) contained 1%-5% xylitol, respectively. The results are summarized in FIG. 6B. No significant change in pH was observed with the new test blends. In fact, the pH rose slightly by the inclusion of xylitol.

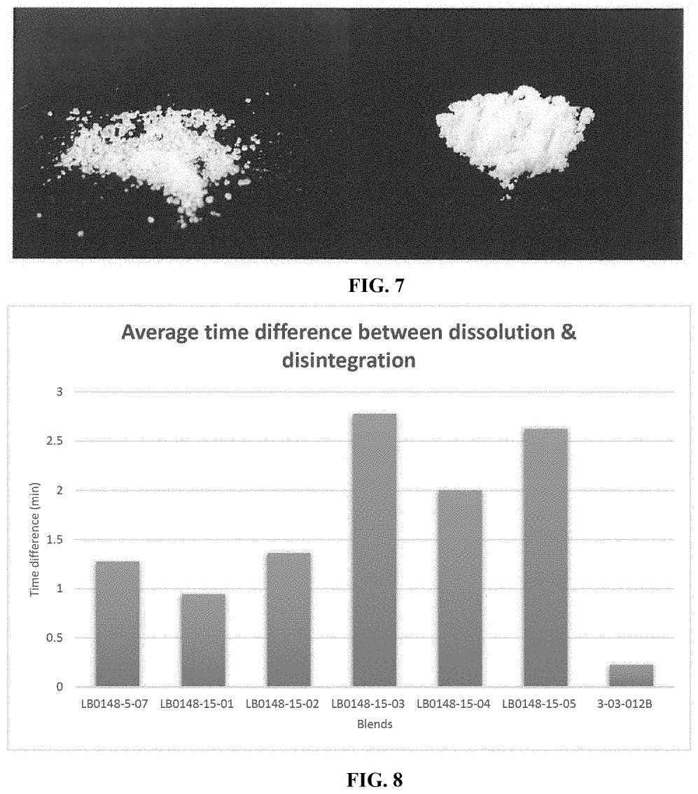

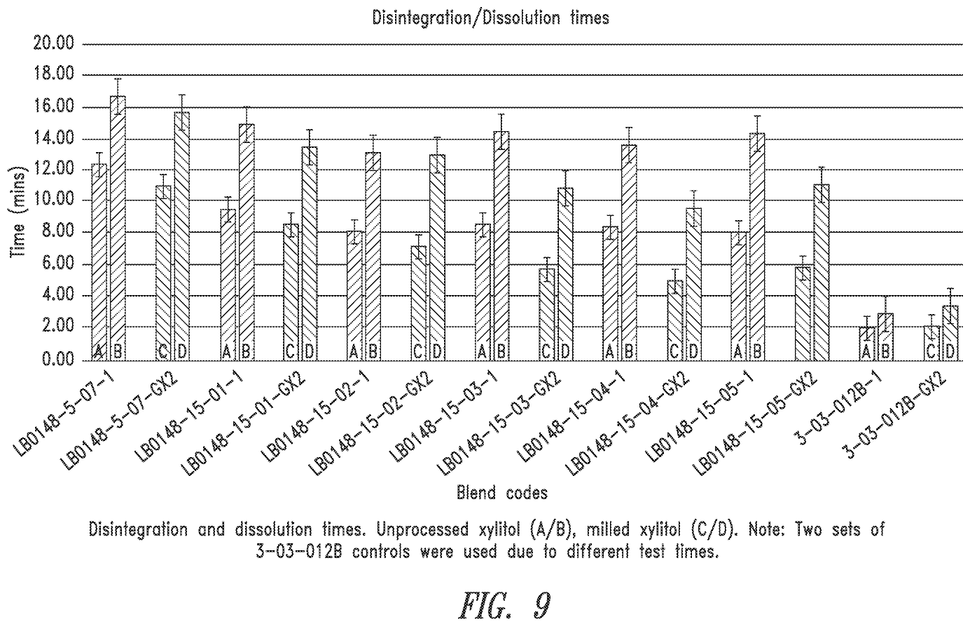

The formulations containing xylitol were then assessed in tablet form. Xylitol was assessed when used unprocessed and in a milled format (FIG. 7). Using unprocessed xylitol, it was observed that the disintegration/dissolution times were increased up to four times compared to Control 1 (3-03-0128). Control 1 (3-03-0128) had an average dissolution time <3 min in 1000 ml of water when tested in the 817 mg tablet format in 1000 ml of water. It is known that in a 49 mg finished weight format, the dissolution of Control 1 (3-03-0128) is <1 min. Additionally, it was noted that as the level of xylitol increased, a longer lag time between dissolution/disintegration was observed (FIG. 8).

When xylitol was ground into a finer powder, disintegration times were decreased for all blends. However, dissolution times saw a 1.5-5 min increase. The exception was Composition 5 ((LB0148-15-05) NaDCC: 17.0%; Adipic acid: 21.0%; Sodium bicarbonate: 34.0%; Sodium carbonate: 23.0%; and Xylitol: 5.0%), which demonstrated a decrease of both disintegration and dissolution times. This formulation had a dissolution time of 8 minutes compared to Control 3 (LB0148-5-07), which had a dissolution time of <3 minutes.

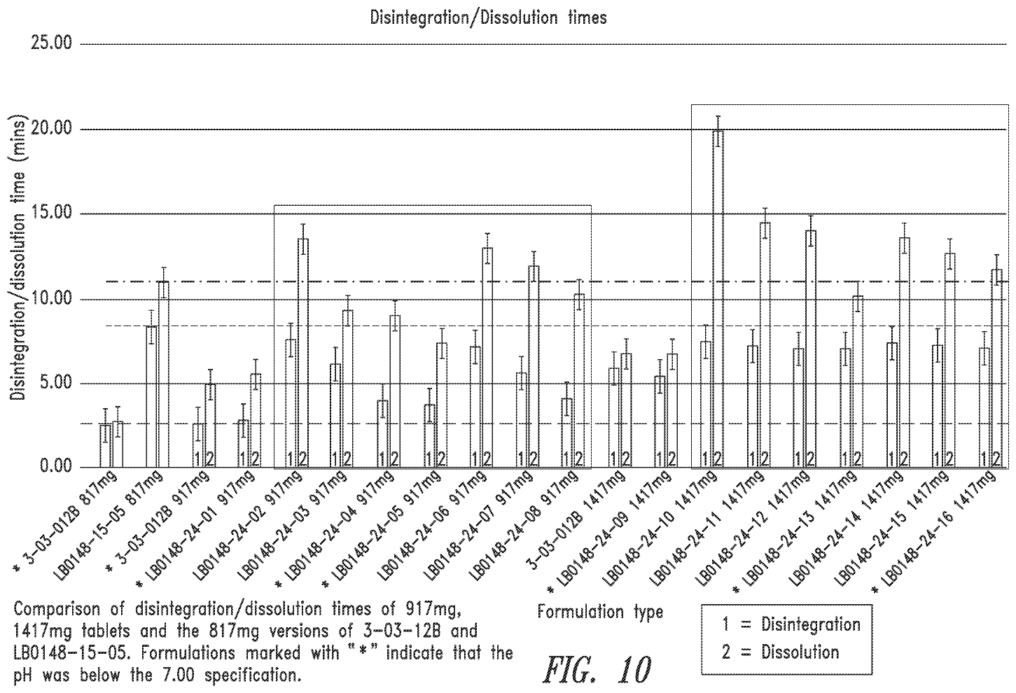

Industrial grade xylitol was also tested in a variety of formulations. Formulations including unprocessed industrial grade xylitol and milled industrial grade xylitol were tested. The results of the 14 tests are summarized in FIG. 9. The chlorine content and pH of the solutions were not impacted by the use of industrial grade xylitol. The dissolution times were slightly improved when milled xylitol was included at a concentration of 3%-5%.

Additional formulations containing up to 10% xylitol were tested. No significant improvement in disintegration or dissolution was noted. Additionally, the pH of the resulting solution was not negatively impacted by the increase in xylitol concentrations.

Formulations containing xylitol concentrations of 3%, 5%, 7%, and 9% were tested using xylitol from three batches having a three different particle sizes. Tablets pressed using the first batch (largest particle size) provided solutions having pH, NaDCC, and chlorine content within the desired range. However, the disintegration time and dissolution time averaged 10 minutes and 15 minutes, respectively. Tablets pressed using the second batch (20-80 mesh) also demonstrated pH, NaDCC, and chlorine content within the desired range, but disintegration time and dissolution time of 11 minutes and 15 minutes, respectively. Tablets pressed using the third batch (120 mesh) maintained pH, NaDCC, and chlorine content within the desired range. The disintegration time was 5 minutes lower on average and the dissolution time was 10 minutes.

Tablet Size

Larger tablet sizes were tested. These formulations contained the same amount of NaDCC per tablet as the 817 mg (corresponding to 80-100 ppm) but with greater amounts of effervescent components. Tablets of 971 mg (corresponding to a 55 mg finished weight) and 1417 mg (corresponding to a final 170 mg finished weight) were tested. Control 1 (3-03-0128) was used as a comparative control. Test formulations were devised using Composition 5 (LB0148-15-05) (containing 5% xylitol). A total of 18 formulations were assessed. A summary of these results is outlined in FIG. 10. While disintegration was improved using a larger tablet size, dissolution was not improved by the larger tablet sizes when pH remained within the range of 7-8.

### Example 2

Tablets including various concentrations of xylitol were tested as indicated in Tables 2 and 3 below.

TABLE 2

| TB4085 (5% xylitol) | Average | Min | Max |
|---|---|---|---|
| Diameter (mm) | 4.78 | 4.76 | 4.82 |
| Thickness (mm) | 1.68 | 1.59 | 1.75 |
| Avg weight (g) | 49.89 | 46.60 | 53.40 |
| Friability | 0% | N/A | N/A |
| Hardness (N) | 26.30 | 18.00 | 39.00 |
| Disintegration 200 mL (mins) | 03:14:00 | 03:08:00 | 03:20:00 |
| Dissolution 1000 mL (mins) | 04:17:00 | 03:39:00 | 04:17:10 |
| NaDCC content (mg/tablet) | 8.00 | N/A | N/A |
| pH in 60 ml | 7.33 | 7.29 | 7.40 |
| Chlorine in 60 ml (ppm) | 94.56 | 78.01 | 106.38 |

TABLE 3

| TB4086 (10% xylitol) | Average | Min | Max |
|---|---|---|---|
| Diameter (mm) | 4.79 | 4.77 | 4.82 |
| Thickness (mm) | 1.76 | 1.70 | 1.85 |
| Avg weight (g) | 50.32 | 47.70 | 52.80 |
| Friability | 4.71% | N/A | N/A |
| Hardness (N) | 28.70 | 17.00 | 37.00 |
| Disintegration 200 mL (mins) | 02:16:10 | 01:53:00 | 02:34:00 |
| Dissolution 1000 mL (mins) | 02:56:30 | 02:22:00 | 03:20:00 |
| NaDCC content (mg) | 9.10 | N/A | N/A |
| pH in 60 ml | 7.02 | 6.92 | 7.10 |
| Chlorine in 60 ml (ppm) | 101.65 | 92.19 | 120.56 |

Although tablets were formed, the tablets failed to release from the molds.

### Example 3

### Materials and Methods

A study is conducted with 30 patients having at least four separate teeth with a pocket depth of at least 5 millimeters (mm) and a minimum number of teeth of at least 20. None of the patients need emergency dental care or revealed systemic conditions that would affect their periodontal status or contraindicate participation in the study. Patients are excluded from the study if they are diabetic, pregnant, immunocompromised, unable to comply with a research protocol, smoked more than 10 cigarettes daily, or received periodontal therapy or systemic antibiotic during six months before entering the study.

For the study, the patients are divided into two randomized groups of 15 patients. The study is a randomized, single-blinded, controlled, clinical trial on parallel groups according to established criteria set by the CONSORT GROUP (Altman D G, Schulz K F, Moher D et al., Consolidated Standards of Reporting Trials) and the revised CONSORT statement for reporting randomized trials: explanation and elaboration (Ann Intern Med 2001; 134:663-694).

The clinical examination is performed by a blinded dental examiner who is calibrated to accomplish more than 90% reproducibility in repeated measurements of clinical variables studied. The patients are also blinded to ensure usage of both control and Composition A mouth rinse.

The first randomized group of patients receives Composition A, which comprises NaDCC and xylitol, in an aqueous solution. The second randomized group of patients receives the control which is water. A syringe tip is placed at the bottom of the pockets of each tooth. The composition in the composition in an aqueous solution or water is repeatedly applied in a circular manner around all teeth for a total of five minutes.

All patients receive three comprehensive clinical full mouth dental examinations. The first examination (visit 1), before the study, provides a baseline for their teeth pocket depth. The second examination (visit 2) is on day 14 of the study and provides a progress report. The third examination (visit 3) is at the conclusion of the study (e.g., three months from the outset) and provides a final result.

The patients fill out a medical questionnaire and receive general oral examinations. The examinations record the full dental radiograph of teeth, and assesses the presence of the dental plaque, gingival bleeding on probing, periodontal pocket depth (in mm) gingival recession (in mm), furcation involvement, and tooth mobility.

At visit 1 and visit 2, the study patients receive clinical examination that includes oral hygiene instruction followed by professional subgingival irrigation. The patients do not receive subgingival or supragingival scaling. The subgingival irrigation is performed using 3 mL monoject and endodontic syringe with a 23 gauge cannula (metal with the blunt and side ports).

The clinical variables of the data from the study are assessed by visual inspection for the presence or absence of supragingival plaque on the facial and lingual surfaces of each tooth.

Before the examination, the teeth surfaces are air dried for enhanced visibility. Visual inspection is further enhanced by indirect vision and light reflection with mouth mirror.

During clinical examination, the results for plaque, gingival bleeding, and probing depth are recorded. A value of “0” is assigned to surfaces with the absence of plaque while value of “1” is assigned to services with the presence of plaque. Bleeding on probing is assessed within 30 seconds after probing to the pool pocket depth, and is recorded on the facial, single, mesiofacial, distofacial, mesiolingual and distolingual surfaces of each tooth. A value of “0” is assigned to teeth showing the absence of gingival bleeding in all six study sites, a value of “1” is assigned to teeth revealing gingival bleeding in any of the six sites. Probing depths are measured in millimeters using probes with probing force of approximately 0.75 N. Pocket depth and gingival recession is measured on the facial, lingual, mesiofacial, distofacial, mesiolingual and distolingual surfaces of each tooth.

Specifically, microbiological sample from two deep periodontitis lesions of each study patient is obtained at all three study visits and analyzed by established anaerobic culture methods (see, e.g., Doan N, Contreras A, Flynn J Morrison J, Slots J. Proficiencies of three anaerobic culture systems of recovering periodontal pathogenic bacteria. J Clin Microbio 1999; 37:171-174). The analysis will include identification of microorganisms including, Aggregatibactor actinomycetemcomitans, Prophyromonas gingivalis, Tannerella forsythia, Prevotella intermedia, Dialister pneumosintes, Fusobacterium species, Campylobacter rectus, Paevimonas micra, Eubacterium species, Beta-hemolytic streptococci, Staphylococci, Gram-negative enteric rods, and Candida species.

At the end of the clinical examination visits 1 and visit 2, the patients receive a manual Oral-B toothbrush and dental floss samples. The patients are instructed to brush twice a day using the modified bass technique and to floss once a day. The patients are also instructed to use the oral rinse at home according to their randomized study groups. Patients are instructed to rinse their mouth every day for one minute with either 1 ounce of Composition A in an aqueous solution or 1 ounce of sterile water.

Patients in the in the treatment test group are provided with Composition A in tablet form. A fresh composition in an aqueous solution is instructed to be made for each rinse by dissolving a tablet of Composition A in one ounce of water. Following rinsing with the composition in an aqueous solution, the test group patients are instructed to expectorate and refrain from rinsing with water for at least 10 minutes. The procedure for making composition in an aqueous solution with water and rinsing with the solution are provided in writing, verbally explained, and practiced under the supervision of the clinicians.

Patients in the control group are supplied mouth rinse containing only water (i.e., with no active ingredients). These patients are given the same instructions as the treatment group to rinse with 1 ounce of the mouth rinse. Following rinsing with water, the control group patients are instructed to expectorate and refrain from rinsing with water for at least 10 minutes. Instructions for using the mouth rinse are provided in writing, verbally explained, and practiced under the supervision of the clinicians.

In an effort to ensure compliance, all study patients are provided with a rinse log to record the exact date and time of rinsing. Additionally, they are instructed to bring the rinse log with them to the final visit. Furthermore, patients are telephoned or sent text regularly to remind them to rinse and to inquire about any problems with the rinsing method. In addition, patients receive an end-of-study written questionnaire to gain evaluated feedback about their satisfaction or concerns with the study.

At the conclusion of the study, each patient receives standard clinical examination for periodontitis, including scaling and root planing, supragingival polishing, and recall schedule to prevent disease recurrence.

### Example 4

Patients (referred to as Patient 1, Patient 2, Patient 3, and Patient 4 below) presenting with symptoms of periodontal disease (e.g., gum inflammation, bleeding on probing, mobility, etc.) were given tablets of a composition that comprises NaDCC, calcium carbonate, and calcium bicarbonate with instructions for use. The patients were instructed to dissolve one tablet in approximately one ounce of water, and to rinse vigorously with the resulting solution for one minute on a daily basis.

Patient 1 used the oral rinse as directed for one month, Patient 2 and Patient 3 used the oral rinse as directed for two weeks, and Patient 4 used the oral rinse as directed for two months

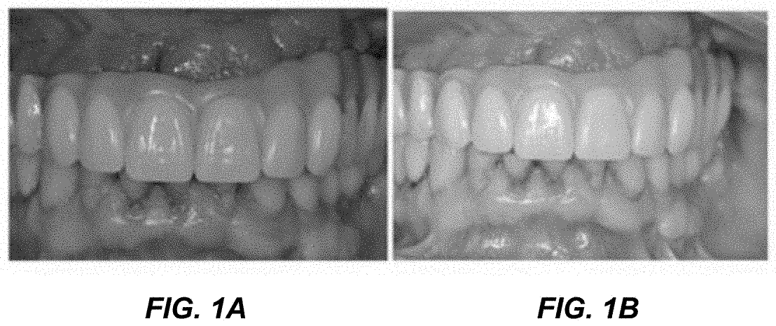

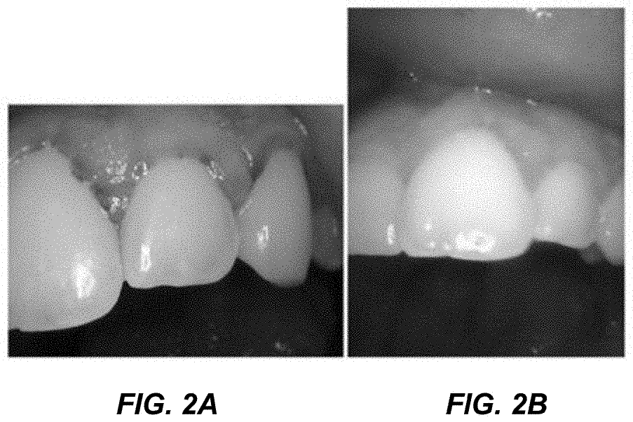

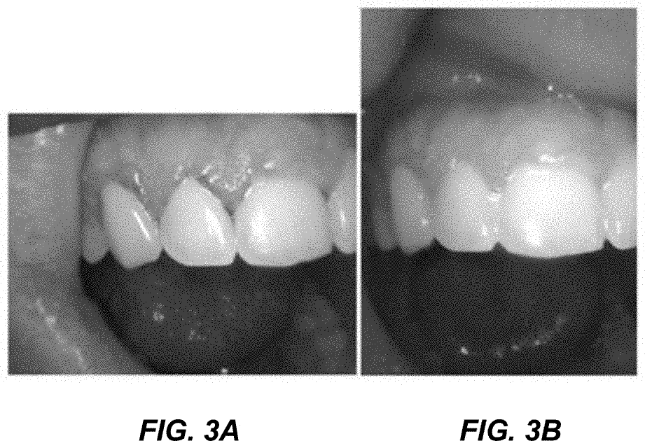

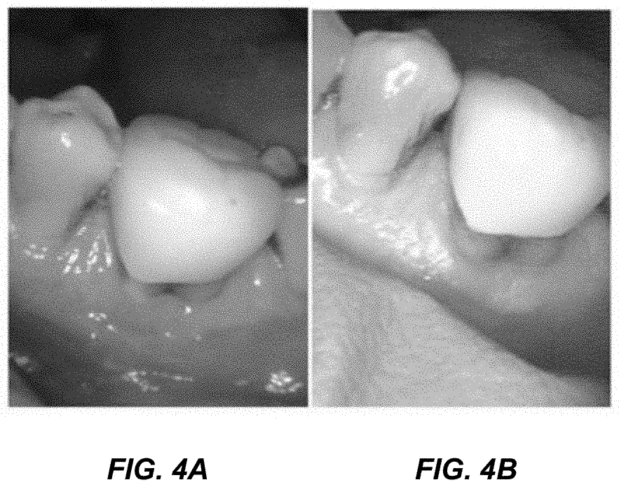

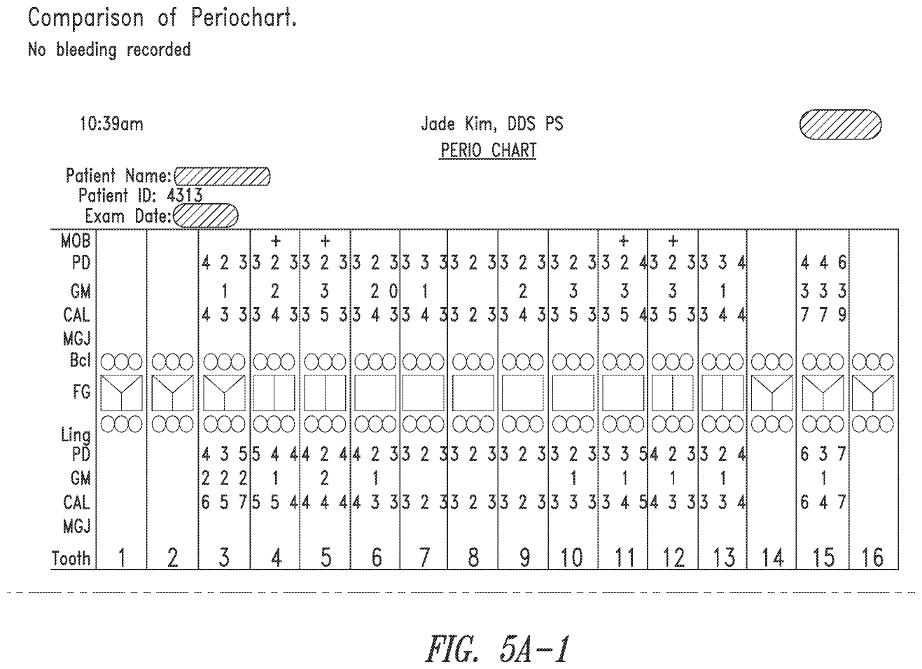

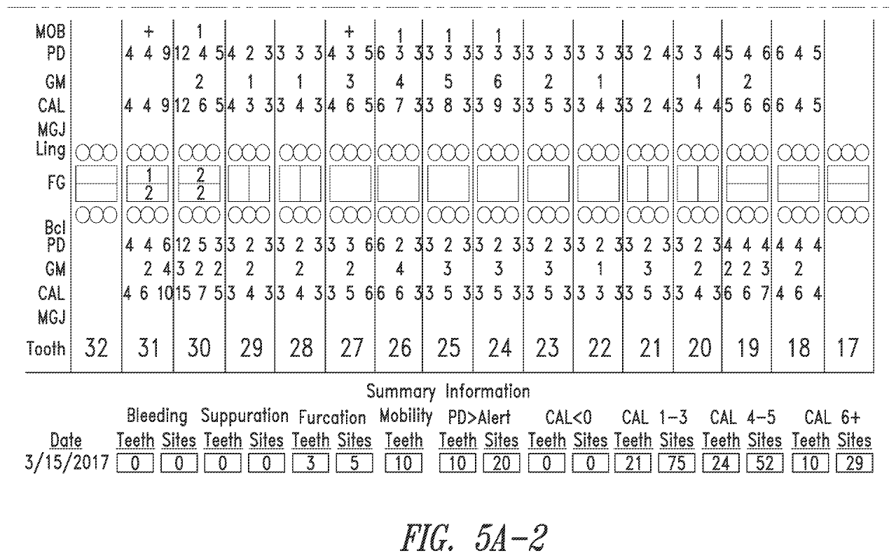

Two photographs were taken of each of the patients. The first photograph was taken at the outset, before using the composition, and the second photograph was taken after using the composition on a daily basis for a set period of time. The initial photographs of Patient 1, Patient 2, Patient 3, and Patient 4 are shown in FIG. 1A, FIG. 2A, FIG. 3A, and FIG. 4A, respectively, and the photographs taken after treatment are shown in FIG. 1B, FIG. 2B, FIG. 3B, and FIG. 4B, respectively. As can be seen in the photographs following treatment, gum redness and inflammation is reduced in all of the patients.

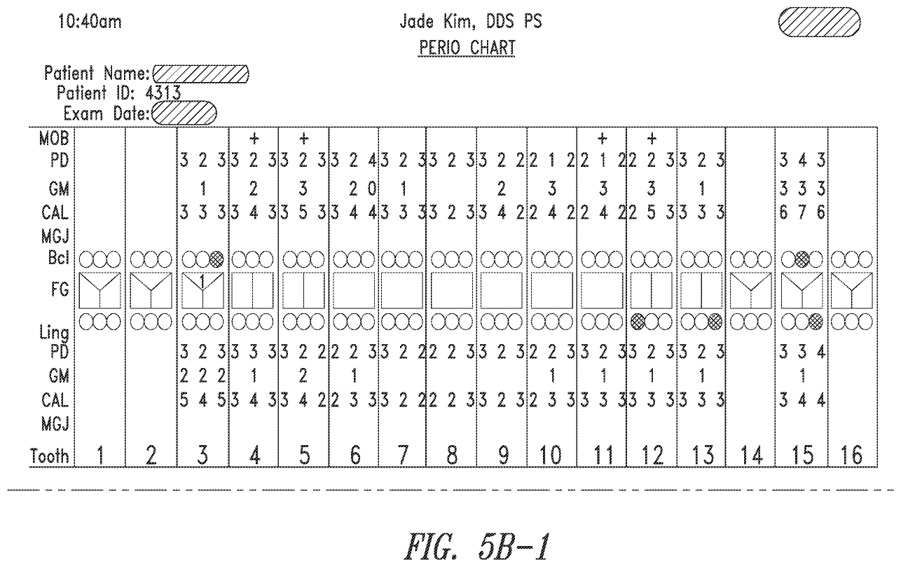

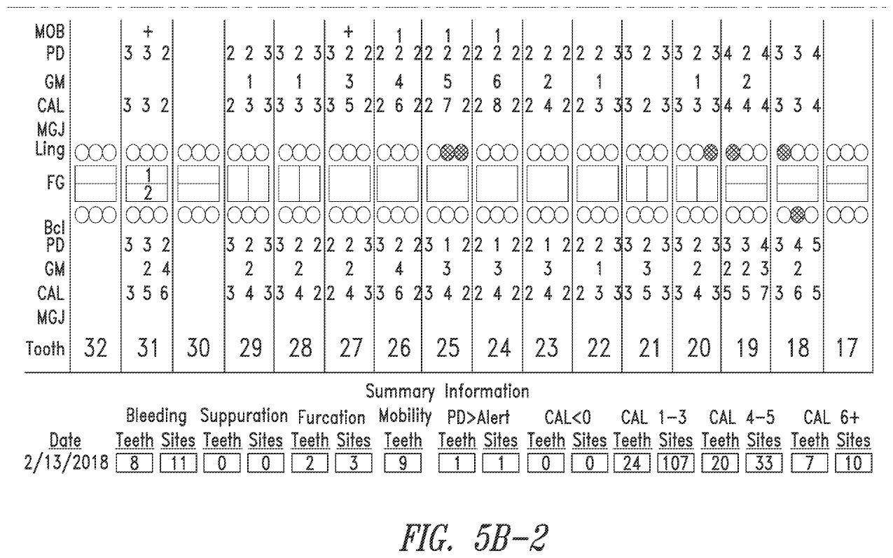

Additionally, FIG. 5A-1 and FIG. 5A-2 show a periodontal chart completed based on an examination prior to treatment with the composition, and FIG. 5B-1 and FIG. 5B-2 show a periodontal chart for the same patient completed after the patient used the composition on a daily basis for 11 months. As can be seen, treatment with the composition resulted in a significant reduction in pocket depth.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition, comprising:
sodium dichloroisocyanurate (NaDCC), or a functional analog thereof;
a sugar substitute selected from Stevia, aspartame, xylitol, or sorbitol, or a functional analog thereof, or a combination thereof;
an alkali metal carbonate selected from calcium carbonate, calcium bicarbonate, sodium carbonate, sodium bicarbonate, or a combination thereof; and
an acid selected from adipic acid, citric acid, tartaric acid, or a combination thereof.

2. The composition of claim 1, wherein the NaDCC is present in an amount ranging from 10 weight percent (wt. %) to 20 wt. %.

3. The composition of claim 1, wherein the sugar substitute is present in an amount ranging from 2 wt. % to 10 wt. %.

4. The composition of claim 1, wherein the alkali metal carbonate is present in an amount ranging from 50 wt. % to 65 wt. %.

5. The composition of claim 1, wherein the alkali metal carbonate is sodium carbonate, sodium bicarbonate, or both.

6. The composition of claim 1, wherein the composition comprises a first alkali metal carbonate and a second alkali metal carbonate.

7. The composition of embodiment 6, wherein the first alkali metal carbonate in an amount ranging from 35 wt. % to 45 wt. %.

8. The composition of claim 6, wherein the second alkali metal carbonate in an amount ranging from 15 wt. % to 20 wt. %.

9. The composition of claim 1, wherein the acid is present in an amount ranging from 20 wt. % to 25 wt. %.

10. The composition of claim 1, further comprising magnesium sulfate, sodium benzoate, or both.

11. The composition of claim 1, wherein the composition is in the form of a tablet.

12. The composition of claim 1, wherein the composition is in the form of a granule or powder, and wherein the granule or powder is in a capsule.

13. The composition of claim 1, wherein the pH of the composition ranges from 7 to 8 when dissolved in 0.5 to three ounces of water.

14. The composition of claim 1, wherein the composition is a solution comprising 0.5 to three ounces of water.

15. The composition of claim 14, wherein the solution comprises at least 100 parts per million (ppm) of free chlorine.

16. The composition of claim 14, wherein the pH of the solution ranges from 7 to 8.

17. A method of manufacturing a composition of claim 1, the method comprising:
combining the NaDCC, or the functional analog thereof, the sugar substitute, the alkali metal carbonate, and the acid into a mixture; and
homogenizing the mixture.

18. A method of controlling bad breath in a subject, the method comprising:
contacting at least a portion of an oral cavity of the subject with an effective amount a composition of claim 1.

19. The method of claim 18, wherein the solution contacts the at least the portion of the oral cavity for 0.5 minutes to two minutes.

20. The method of claim 18, further comprising expelling the composition after the contacting.
